(19) United States
(12) Patent Application Publication
(10) Pub. No.: US 2015/0355179 A1
(43) Pub. Date: Dec. 10, 2015

(54) CDKACT BIOSENSORS-FLUORESCENT POLYPEPTIDE TO PROBE THE ACTIVITY OF CDK/CYCLIN KINASES IN VITRO, IN CELLULO AND IN VIVO

(71) Applicant: CENTRE NATIONAL DE LA RECHERCHE SCIENTIFIQUE (CNRS), Paris (FR)

(72) Inventors: May Catherine Morris, Mauguio (FR); Ngoc Van, Montpellier (FR); Morgan Pellerano, Aigues-Vives (FR)

(73) Assignee: CENTRE NATIONAL DE LA RECHERCHE SCIENTIFIQUE (CNRS), Paris (FR)

(21) Appl. No.: 14/433,934

(22) PCT Filed: Oct. 10, 2013
(86) PCT No.: PCT/EP2013/071179

§ 371 Date: Apr. 7, 2015

(30) Foreign Application Priority Data
Oct. 10, 2012 (EP) 12306239.0

(51) Int. Cl. G01N 33/573; G01N 33/58; G01N 33/542; C07K 7/08

(52) U.S. Cl. CPC G01N 33/573; C07K 7/08; G01N 33/581; G01N 33/542; G01N 2333/91205; A61K 38/00

(57) ABSTRACT

This invention relates to compounds comprising a polypeptide and a fluorophore, wherein the polypeptide is capable of being phosphorylated by a Cyclin-Dependent Kinase (CDK) and/or a Cyclin-CDK complex on a specific site and wherein the fluorescence of said fluorophore changes upon phosphorylation of said specific site of said polypeptide. The invention also relates to the use of such compounds or of compositions comprising such compounds for medical imaging. The invention furthermore relates to methods for determining if at least one CDK and/or Cyclin-CDK complex is active, for determining changes in at least one CDK and/or Cyclin-CDK complex activity, for the in vitro diagnosis of Cyclin-dependent kinase hyperactivation in a subject.

This invention relates to compounds comprising a polypeptide and a fluorophore, wherein the polypeptide is capable of being phosphorylated by a Cyclin-Dependent Kinase (CDK) and/or a Cyclin-CDK complex on a specific site and wherein the fluorescence of said fluorophore changes upon phosphorylation of said specific site of said polypeptide. The invention also relates to the use of such compounds, or of compositions comprising such compounds, for medical imaging. The invention furthermore relates to methods for determining if at least one CDK and/or Cyclin-CDK complex is active, for determining changes in at least one CDK and/or Cyclin-CDK complex activity or for the in vitro diagnosis of Cyclin-dependent kinase hyperactivation in a subject.

Cell cycle progression is driven by a family of serine/threonine protein kinases named Cyclin-Dependent Kinases (CDK), whose sequential activities promote phosphorylation of key substrates involved in cell growth and division (Malumbres et al., 2005; Obaya et al., 2002; Satyanarayana et al., 2009; Merrick et al., 2010). Cyclin-CDK complexes are formed through association of a CDK with a Cyclin partner, which plays a major role in promoting activation of the CDK by inducing significant conformational changes, in defining substrate specificity and in targeting the heterodimeric complex to well-defined subcellular locations (Jeffrey et al., 1995; Morgan et al., 1997; Morris et al., 2002; Lolli et al., 2010). The kinase activity of CDK and/or Cyclin-CDK complexes is primarily conditioned by formation of the Cyclin-CDK complex, and thus expression of either counterpart. This heterodimeric complex is then further regulated by several phosphorylations on the CDK, that either inhibit or promote its complete activation (Morgan et al., 1997). Additional regulatory proteins are known to regulate CDK and/or Cyclin-CDK complex activity, such as the INK4 family and the Cip/Kip family of CKI (Cyclin-dependant Kinase Inhibitors).

CDK and/or Cyclin-CDK complex activities are frequently altered in human cancers and contribute to sustain abnormal proliferation in cancer cells (Lapenna et al., 2009; Malumbres et al., 2009). More particularly, aberrant CDK and/or Cyclin-CDK complex activity have been reported in a wide range of cancers including breast, ovarian, prostate, colorectal and lung cancer, lymphoma, myeloma and sarcoma (Harwell et al., 2004; Ekberg et al., 2005; Husdal et al., 2006; Suzuki et al., 2007; Kim et al., 2009). CDK and/or Cyclin-CDK complex aberrant activity may result from many different causes, such as gene amplification, protein overexpression, mislocalization, expression of truncated variants, or posttranslational modifications affecting either Cyclins, CDK or regulatory proteins such as the INK4 family and the Cip/Kip family of CKI (Stivala et al., 2012; Nozoe et al., 2006). For example, a subset of mutations of CDK4 and CDK6 are known to confer a selective growth advantage through loss of natural inhibitor (CKI) binding, whilst other mutations have been reported to promote CDK1, CDK2 or CDK4 overexpression (Malumbres et al., 2001; Malumbres at al., 2007). Because of this diversity of causes, and despite its oncological relevance, there are no direct means of assessing CDK and/or Cyclin-CDK complexes activity, particularly in real-time and/or in living cells.

Indeed, the measure of CDK and/or Cyclin-CDK complexes activity remains essentially limited to assays based on radioactivity incorporation or on antigenic approaches. Most of these studies have traditionally been performed using biochemical assays based on purified enzymes produced as recombinant proteins from insect or mammalian cells in culture. These assays have been widely used and adapted to high-throughput drug screening, but they are endpoint assays, which no not allow for real-time analysis of kinase activity, are not reversible and further lack the physiological context of the cell.

Cell-based methods that monitor kinase activity, for example in the presence of a potential drug candidate, have been developed that rely on the incorporation of ³²P into cells. Following ³²P incorporation and incubation in the presence of a drug candidate, the cells are lysed and the substrate protein is isolated and purified to determine its relative degree of phosphorylation by measuring the amount of ³²P incorporated. Such cell-based assays are labor intensive and only poorly sensitive, and have the disadvantage of requiring high levels of radioactivity. Other cell-based assays for the study of kinase activity use radiolabeled phosphorylation-specific antibodies (i.e., antibodies that can distinguish between phosphorylated and non-phosphorylated proteins). In these assays, the phosphorylated substrate protein is detected and quantified by immunoprecipitation, gel electrophoresis or Western blotting after lysis of the cells. Although these assays generally require lower levels of radioactivity than ³²P-based methods, they are equally labor intensive, time consuming and complex to automate.

Non-radioactive cell-based methods have emerged that use an ELISA (i.e., enzyme-linked immunosorbent assay) approach to measure the activation of specific kinase signaling pathways. These kinase assays, which employ phosphorylation-specific antibodies, have been demonstrated to be suitable for high-throughput drug screening (Versteeg et al., 2000).

Overall, the existing assays require cell lysis, which implies that any corresponding read-outs will represent an average for CDK and/or Cyclin-CDK complexes activity states across the entire cell population(s) studied. Such averaging does not allow potential differences or variations between individual cells to be detected and therefore may mask significant biological information on the distribution of CDK and/or Cyclin-CDK complexes activity within a cell population. Moreover, data collected for real-time analysis of CDK and/or Cyclin-CDK complexes activity with those assays are biased by the necessity to use as many samples as there are read-outs.

Non-radioactive high-throughput screening methods have been developed (Kupcho et al., 2003), based on the use of fluorogenic peptide substrates (Rhodamine 110, bis peptide amide) that are cleaved before phosphorylation to release the free Rhodamine 110; upon phosphorylation, cleavage is hindered, and the compound remains as a nonfluorescent peptide conjugate. While those methods do not involve cell lysis, they do not allow for any real-time analysis, as the non-phosphorylated substrates are cleaved and degraded.

Improved methods are still needed for the qualitative and quantitative assessment of CDK and/or Cyclin-CDK complex activity, as well as for the identification of modulators of such activity under conditions that most closely mimic the actual in vivo situation. In particular, cell-based assays that are simple, rapid, sensitive and adaptable to high-throughput screening, that provide information about individual cells within a cell population, and that allow the potential therapeutic value of candidate compounds to be evaluated in the early phases of the drug discovery and development process are highly desirable.

The inventors have designed a compound comprising a polypeptide and a fluorophore, whose fluorescence increases in a sensitive fashion upon phosphorylation of the peptide by CDK and/or Cyclin-CDK complexes in a sensitive and reversible fashion. The compound can be used to assess the activity of CDK and/or Cyclin-CDK complexes, through fluorescence imaging. Additionally, the inventors have found that the compound may be used with living cells or tissues, for example following cell delivery with a cell-penetrating peptide. The inventors have set up methods that allow for the detection of subtle differences in CDK and/or Cyclin-CDK complex activity between different cell lines in a standardized and sensitive, yet non-destructive fashion. Those compounds and methods afford direct readout and real-time monitoring of CDK and/or Cyclin-CDK complexes activity either in extracts or in living cells, thus providing tools to identify cells or tissues in which these CDK and/or Cyclin-CDK complexes are hyperactive, for cancer diagnostics, for monitoring response to therapeutics, and for cell-based drug discovery strategies.

The compound of the invention is based on the strong fluorescence enhancement exhibited by fluorophores, particularly environmentally-sensitive dyes, when their exposure to their immediate environment is modified. Upon phosphorylation by a CDK and/or Cyclin-CDK complexes the compound of the invention undergoes a conformational change, which substantially modifies the immediate environment of the fluorophore. The emitted fluorescence of the compound thus varies with its phosphorylation state, and particularly increases substantially when it is phosphorylated by a CDK and/or Cyclin-CDK complexes. These variations are reversible, and thus afford for real-time analysis of CDK and/or Cyclin-CDK complex activity.

### DETAILED DESCRIPTION OF THE INVENTION

The inventors have designed compounds comprising a polypeptide and a fluorophore, wherein the polypeptide is capable of being phosphorylated by a CDK and/or a Cyclin-CDK complex on a specific site and wherein the fluorescence of said fluorophore changes upon phosphorylation of said specific site of said polypeptide.

Each amino acid is herein represented according to the IUPAC amino-acid abbreviation, such as follows:

TABLE 1

| Amino-acid or | | |
|---|---|---|
| amino-acid residue | Abbreviation | Abbreviation |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Aspartic acid or Asparagine | Asx | B |
| Glutamine or Glutamic acid. | Glx | Z |
| Any amino acid. | Xaa | X |

This invention thus firstly relates to compounds comprising a polypeptide and at least one fluorophore, characterized in that:
a. said polypeptide comprises:

i. a substrate domain comprising the sequence X₁-S₁-S₂/T-P-X₂ (SEQ ID N^{o} 30) and capable of being phosphorylated by a CDK and/or a cyclin-CDK complex on the amino acid residue at position 3 of said sequence SEQ ID N^{o} 30,

ii. a phosphobinding domain,

iii. wherein said phosphobinding domain and said substrate domain are capable of interacting upon the phosphorylation of said S₂ amino-acid residue or of said T amino-acid residue of said sequence X₁-S₁-S₂/T-P-X₂ in the substrate domain,

iv. wherein the substrate domain and the phosphobinding domain are linked by a linker domain, and
b. said at least one fluorophore is coupled to an amino-acid residue of said substrate domain.

In a particular embodiment, the present invention relates to a compound comprising a polypeptide and only one fluorophore. According to this particular embodiment, the change in fluorescence emission of said only one fluorophore is monitored.

In a particular embodiment, the present invention relates to a compound comprising a polypeptide and at least one fluorophore, wherein said compound is an isolated compound. By “isolated compound” it is intended a compound which is separated, by purification, or partial purification, from its environment.

In another particular embodiment, the present invention relates to a compound comprising a polypeptide and at least one fluorophore, wherein said polypeptide is an isolated polypeptide. By “isolated polypeptide” it is intended a polypeptide which is obtained by a method known by a person skilled in the art, including chemical synthesis and recombinant production, said polypeptide being subsequently separated, by purification or partial purification, from its initial environment.

According to the invention, the substrate domain is a polypeptide comprising the sequence X₁-S₁-S₂/T-P-X₂ (SEQ ID N^{o} 30) and capable of being phosphorylated by a CDK and/or a Cyclin-CDK complex on the amino acid residue at position 3 of said sequence; said amino-acid residue at position 3 of said sequence X₁-S₁-S₂/T-P-X₂ (SEQ ID N^{o} 30) being either the serine S₂ residue or threonine T residue, and wherein X₁ and X₂ are any natural amino acid residue, particularly any of the amino acid residues listed in Table 1.

In a particular embodiment, the invention relates to a compound comprising a polypeptide and at least one fluorophore, characterized in that, in the substrate domain of said polypeptide, said amino-acid X₁, at position 1 of SEQ ID N^{o} 30, is a cysteine. According to said embodiment, a cysteine residue is located at position −2 relatively to the amino-acid at position 3 of SEQ ID N^{o} 30.

In another particular embodiment, the invention relates to a compound comprising a polypeptide and at least one fluorophore, characterized in that, in the substrate domain of said polypeptide, said amino-acid X₂, at position 1 of SEQ ID N^{o} 30, is a cysteine.

In another particular embodiment, the invention relates to a compound comprising a polypeptide and at least one fluorophore, characterized in that, in the substrate domain of said polypeptide, said amino-acid X₁, at position 1 of SEQ ID N^{o} 30 and said amino-acid X₂, at position 5 of SEQ ID N^{o} 30 are a cysteine. According to said embodiment, a cysteine residue is located at position −2 relatively to the amino-acid at position 3 of SEQ ID N^{o} 30.

In an embodiment, the substrate domain is capable of being phosphorylated by a Cyclin-dependent kinase (CDK) chosen in the list consisting of: CDK1, CDK2, CDK3, CDK4, CDK5, CDK6, CDK7, CDK8, CDK9 or CDK10. In a preferred embodiment, the substrate domain is capable of being phosphorylated by at least one CDK chosen in the list consisting of: CDK1, CDK2, CDK3, CDK4, CDK5, CDK6, CDK7, CDK8, CDK9, CDK10. In a more preferred embodiment, the substrate domain is capable of being phosphorylated by only one of the CDK chosen in the list consisting of: CDK1, CDK2, CDK3, CDK4, CDK5, CDK6, CDK7, CDK8, CDK9 and CDK10.

As disclosed herein, “capable of being phosphorylated” means that a phosphate group may be covalently transferred on said substrate by a CDK and/or by a cyclin-CDK complex. The capacity of a substrate according to the invention of being phosphorylated may, for example, be assessed by a radioactive kinase assay, such as described in the Example 1 of the present Application. The products of such an assay may be analysed by using techniques well known by a person skilled in the art, and for example by gel electrophoresis and immunolabelling, as described in FIG. 1C.

The substrate specificity of a CDK is regulated by its Cyclin counterpart, and it is known by the person skilled in the art that different Cyclin-CDK complexes comprising the same CDK may have different substrates. The skilled person may decide, for example, to provide more specificity to the compound of the invention, to select a substrate domain which is capable of being specifically phosphorylated by at least one CDK wherein said CDK is complexed with a Cyclin. In a particular embodiment, the substrate domain is capable of being phosphorylated by at least one Cyclin-CDK complex. In a preferred embodiment, the substrate domain is capable of being phosphorylated by at least one of the Cyclin-CDK complex chosen in the list consisting of the list of: Cyclin A—CDK1, Cyclin B—CDK1, Cyclin A—CDK2, Cyclin E—CDK2, Cyclin C—CDK3, Cyclin D1—CDK4, Cyclin D2—CDK4, Cyclin D3—CDK4, Cyclin D1—CDK6, Cyclin D2—CDK6, Cyclin D3—CDK6, Cyclin H—CDK7, Cyclin C—CDK8, Cyclin T1—CDK9, Cyclin T2a—CDK9, Cyclin T2b—CDK9, Cyclin K—CDK9. The skilled person may decide to choose specifically a substrate domain which is capable of being phosphorylated by only one Cyclin-CDK complex. In a particular embodiment, the substrate domain is capable of being phosphorylated by only one of the Cyclin-CDK complex chosen in the list consisting of the list of: Cyclin A—CDK1, Cyclin B—CDK1, Cyclin A—CDK2, Cyclin E—CDK2, Cyclin C—CDK3, Cyclin D1—CDK4, Cyclin D2—CDK4, Cyclin D3—CDK4, Cyclin D1—CDK6, Cyclin D2—CDK6, Cyclin D3—CDK6, Cyclin H—CDK7, Cyclin C—CDK8, Cyclin T1—CDK9, Cyclin T2a—CDK9, Cyclin T2b—CDK9, Cyclin K—CDK9.

The selection of a substrate domain according to the invention can be realized by any method known by the person skilled in the art for the evaluation of the phosphorylation of a polypeptide by a kinase, for example by incubating in vitro an immunoprecipitated kinase, in the context of the present invention, a CDK and/or a Cyclin-CDK complex, with said polypeptide in the presence of ATP. Measurement of the phosphorylated polypeptide can be assessed by several reporter systems including colorimetric, radioactive, or fluorometric detection.

Some examples of such tests or assays are disclosed in the example section of the present description, for illustration purposes, without limiting the method of selection of the substrate domain according to these examples.

In a preferred embodiment, the amino acid sequence of the substrate domain of a polypeptide according to the invention comprises an amino acid sequence derived from the sequence of at least one the protein chosen from the following: histone H1, Cyclin B1, lamin B2, Rb (Retinoblastoma protein), and Tau, it further comprises the sequence X₁-S₁-S₂/T-P-X₂ (SEQ ID N^{o} 30) and is capable of being phosphorylated by a CDK and/or a Cyclin-CDK complex on the amino-acid residue at position 3 of said sequence SEQ ID N^{o} 30; said amino-acid residue on position 3 of said X₁-S₁-S₂/T-P-X₂ sequence being either the serine S₂ residue or threonine T residue, and wherein X₁ and X₂ are natural amino acid residue, particularly any of the amino acid residues listed in Table 1.

In a more preferred embodiment, the substrate domain has a sequence derived from at least one of the sequences chosen from:

(SEQ ID No 1)
a. GGCSTPKKAKKL,
(SEQ ID No 2)
b. PEPILVDCSSPSPMET,
(SEQ ID No 3)
c. RAGGPATCSSPTRL,
(SEQ ID No 4)
d. YKFCSSPLRIPG,
(SEQ ID No 5)
e. SGYSSPGSCSTPGSR,

and comprises the sequence X₁-S₁-S₂/T-P-X₂ (SEQ ID N^{o} 30) and is capable of being phosphorylated by a CDK and/or a Cyclin-CDK complex on said serine S₂ residue and/or threonine T residue of said sequence X₁-S₁-S₂/T-P-X₂ (SEQ ID N^{o} 30), wherein X₁ and X₂ can be any natural amino acid residue, particularly any of the amino acid residues listed in Table 1, and more particularly is a cysteine.

As used herein, a “derivative” or “sequence derived from” refers to an amino acid sequence having at least 70% identity with the reference amino acid sequence, preferably at least 80% identity, and most preferably at least 90% identity.

As used herein the term “identity” herein means that two amino acid sequences are identical (i.e. at the amino acid by amino acid basis) over the window of comparison. The term “percentage of sequence identity” is calculated by comparing two optimally aligned sequences over the window of comparison, determining the number of positions at which the identical amino acid residues occurs in both sequences to yield the number of matched positions, dividing the number of matched positions by the total number of positions in the window of comparison (i.e. the window size) and multiplying the result by 100 to yield the percentage of sequence identity. The percentage of sequence identity of an amino acid sequence can also be calculated using BLAST software with the default or user defined parameter.

In a particular embodiment, the invention relates to a compound comprising a peptide, wherein the amino-acid sequence of the substrate domain of said peptide comprises at least one of the sequences chosen from the list consisting of:

(SEQ ID No 1)
a. GGCSTPKKAKKL
(SEQ ID No 2)
b. PEPILVDCSSPSPMET
(SEQ ID No 3)
c. RAGGPATCSSPTRL
(SEQ ID No 4)
d. YKFCSSPLRIPG
(SEQ ID No 5)
e. SGYSSPGSCSTPGSR.

According to the invention, the substrate domain has a sequence that comprises at least 5 amino-acid residues. In an embodiment, the substrate domain of the invention has a sequence that comprises less than 100 amino-acid residues, preferably less than 50 amino-acid residues, even more preferably less than 25 amino-acid residues.

In another embodiment, the present invention relates to a compound comprising a peptide and a fluorophore, wherein the amino-acid sequence of the phosphobinding domain of the peptide has a sequence derived from the sequence of any protein capable of binding specifically to the substrate domain when serine S₂ residue and/or threonine T₁ residue of said X₁-S₁-S₂/T-P-X₂ sequence of said substrate domain is phosphorylated by a Cyclin-CDK complex.

In a more particular embodiment, the phosphobinding domain of a polypeptide according to the invention has a sequence derived from a sequence chosen among the group consisting of: the sequence of human Plk1 (SEQ ID N^{o} 6), the sequence of Pin1 (SEQ ID N^{o} 7), the sequence of Chk2 (SEQ ID N^{o} 8). A person skilled in the art will easily be able to identify the sequences of these proteins in protein sequence databases. In particular, the amino acid sequence of the human Serine/Threonine kinase Plk1 is identified under accession number P53350 in SwissProt database.

In a preferred embodiment, the phosphobinding domain according to the invention has a sequence derived from the phosphobinding domain of Plk1 (SEQ ID N^{o} 9), the WW domain of Pin1 (SEQ ID N^{o} 10) or the FHA domain of Chk2 (SEQ ID N^{o} 11).

In an even more particular embodiment, the phosphobinding domain according to the invention has a sequence derived from the phosphobinding domain of Plk1 (SEQ ID N^{o} 9), wherein at least one cysteine residue is modified for a residue chosen in the list consisting of glycine, alanine, valine, leucine, or serine residue. Preferably, the phosphobinding domain of a polypeptide according to the invention has a sequence derived from the phosphobinding domain of Plk1 (SEQ ID N^{o} 9), wherein all the cysteine residues are modified for a residue chosen in the list consisting of glycine, alanine, valine, leucine, and serine residue. More preferably, the phosphobinding domain according to the invention has the sequence (SEQ ID N^{o} 12).

In another embodiment, the phosphobinding domain according to the invention has a sequence derived from the FHA domain of Chk2 (SEQ ID N^{o} 11), wherein at least one cysteine residue is modified for a residue chosen in the list consisting of glycine, alanine, valine, leucine, and serine residues. Preferably, the phosphobinding domain according to the invention has a sequence derived from the FHA domain of Chk2 (SEQ ID N^{o} 11), wherein all the cysteine residues are modified for a residue chosen in the list consisting of glycine, alanine, valine, leucine, or serine residue. More preferably, the phosphobinding domain according to the invention has the sequence (SEQ ID N^{o} 13).

In another embodiment the sequence of the phosphobinding domain according to the invention comprises a sequence chosen from the list consisting of the sequence SEQ ID N^{o} 10, the sequence SEQ ID N^{o} 12, the sequence SEQ ID N^{o} 13.

According to the invention, the phosphobinding domain has a polypeptide sequence that comprises at least 5 amino-acid residues. Advantageously, the phosphobinding domain of the invention has a polypeptide sequence that comprises less than 300 amino acids, preferably less than 100 amino-acid residues, preferably less than 50 amino-acid residues, even more preferably less than 25 amino-acid residues.

According to the invention, the linker domain is a peptide sequence bound to both the substrate sequence and to the phosphobinding domain sequence.

According to the invention, the linker domain has a polypeptide sequence that comprises less than 50 amino-acid residues, preferably less than 30 amino-acid residues, more preferably less than 15 amino-acid residues and even more preferably at least 2 amino-acid residues.

Preferably, the linker according to the invention comprises at least one proline residue and/or one glycine residue. In an embodiment, the linker domain according to the invention derives from the sequence PGAGGTGGLPGG (SEQ ID N^{o} 14). In a preferred embodiment, the linker domain according to the invention has the sequence PGAGGTGGLPGG (SEQ ID N^{o} 14).

According to the invention, the fluorophore coupled to an amino-acid residue of the polypeptide substrate domain is any fluorescent molecule. By fluorophore, it is herein meant a molecule capable of re-emitting light upon light excitation, or other electromagnetic light. In most cases, emitted light has a longer wavelength, and therefore lower energy, than the absorbed light. Fluorophores typically contain several combined aromatic groups, or plane or cyclic molecules with several π bonds.

According to the invention, the fluorophore is for example chosen from Xanthene, Cyanine, Naphthalene, Coumarin, Oxadiazole, Pyrene, Oxazine, Acridine, Arylmethine or Tetrapyrrole derivatives. The fluorophore chosen from the Xanthene derivatives is for example fluorescein, rhodamine, Oregon green, eosin, or Texas red. The fluorophore chosen from the Cyanine derivatives is for example: cyanine, indocarbocyanine, oxacarbocyanine, thiacarbocyanine, or merocyanine. The fluorophore chosen from the Naphthalene derivatives is for example dansyl or prodan. The fluorophore chosen from the Oxadiazole derivatives is for example pyridyloxazole, nitrobenzoxadiazole or benzoxadiazole. The fluorophore chosen from the Pyrene derivatives is for example cascade blue. The fluorophore chosen from the Oxazine derivatives is for example: Nile red, Nile blue, cresyl violet, or oxazine 170. The fluorophore chosen from the Acridine derivatives is for example proflavin, acridine orange, or acridine yellow. The fluorophore chosen from the Arylmethine derivatives is for example auramine, crystal violet, or malachite green. The fluorophore chosen from the Tetrapyrrole derivatives is for example porphin, phtalocyanine, or bilirubin.

Preferably, the fluorophore according to the invention is chosen in the list consisting of Hydroxycoumarin, Aminocoumarin, Methoxycoumarin, Cascade Blue, Pacific Blue, Pacific Orange, Lucifer yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhodamine B, Texas Red, Allophycocyanin (APC), APC-Cy7 conjugates.

The inventors have found that the change in fluorescence emission upon binding of the phosphobinding domain to the substrate domain when serine S₂ residue and/or threonine T residue of said X1-S₁-S₂/T-P-X₂ sequence of said substrate domain is phosphorylated is more easily monitored and allows for a more accurate and sensitive measure of said binding when using fluorophores that are for example environment-sensitive dies or couples of fluorescent dyes capable of FRET.

In an embodiment of the invention, at least one fluorophore is an environment-sensitive dye. The terms “environment-sensitive dye”, “environment-sensitive probe”, “solvatochromic dye”, “solvatochromic probe” are herein interchangeable. By environment-sensitive dye, it is herein meant a fluorophore the properties of which change, for example intensity, half-life, and excitation or emission spectra, in a measureable manner upon a change in the fluorophore environment. Preferably, by environment-sensitive dye, it is herein meant a fluorophore the intensity or emission spectrum of which changes upon a change in its environment. According to the invention, the change in the fluorophore environment may be due to at least one of a variety of different environmental factors, such as polarity or hydrophobicity. Environment-sensitive have been reviewed in Loving et al., (2010) and Lowder et al., 2011.

Environment-sensitive dyes are well known by the skilled person and may include for example any dye that contains an electron-donating and an electron-accepting group at opposite ends of the aromatic system. According to the invention, the environment-sensitive dye is for example, without restriction to those examples, Cascade Yellow, prodan, Dansyl, Dapoxyl sulfonic acid, NBD, PyMPO, Pyrene, diethylaminocoumarin, SYPRO Orange dye, SYPRO Red dye, nile red, CPM (7-Diethylamino-3-(4′-Maleimidylphenyl)-4-Methylcoumarin), DCDHF (2,7-Dichlorodihydrofluorescein diacetate), fluorophore from the BODIPY family of dyes (boron-dipyrromethene family of dyes).

In another embodiment, the compound according to the invention comprises a polypeptide and at least a couple of fluorescent dyes capable of FRET.

According to the invention, FRET (Förster resonance energy transfer) is a property in which the energy of the excited electron of one fluorophore, called the donor, is passed on to a nearby acceptor dye, resulting in a reduced fluorescence. For the purpose of the invention, the terms “fluorescence resonance energy transfer” and “Förster resonance energy transfer” are equivalent.

According to the invention, a couple of fluorescent dyes capable of FRET means two fluorescent dyes F1 and F2, wherein F₁ is a first fluorophore and F₂ is a second fluorophore, characterized in that F1 is a donor dye and F2 is an acceptor dye, and wherein F2 has an excitation spectrum which overlaps with the emission spectrum of the donor dye F1.

Couples of fluorescent dyes capable of FRET (Förster resonance energy transfer) upon light excitation are well known by the skilled person and may include for example the donor-acceptor couples: cyan fluorescent protein (CFP)-yellow fluorescent protein (YFP) IAEDANS-Fluorescein, EDANS-Dabcyl, Fluorescein-Fluorescein, BODIPY FL-BODIPY FL, Fluorescein-QSY 7 or Fluorescein-QSY 9.

In some embodiments, the donor and acceptor dyes are different, in which case FRET can be detected by the appearance of sensitized fluorescence of the acceptor or by quenching of donor fluorescence. When the donor and acceptor are the same, FRET can be detected by the resulting fluorescence depolarization.

According to the invention, the fluorophore is coupled to specific functional groups, for example specific functional groups of amino-acid residues, such as amino, carboxyl, thiol or azide groups. In a preferred embodiment, the fluorophore is coupled to a thiol group of an amino-acid residue. In a more preferred embodiment, the fluorophore is coupled to a thiol group of a cysteine residue. Coupling the fluorophore to an amino acid functional group is a technique well known by the skilled person, and may involve chemical reactions such as for example amine coupling of lysine amino acid residues (typically through amine-reactive succinimidyl esters), sulfhydryl coupling of cysteine residues (via a sulfhydryl-reactive maleimide) or photochemically initiated free radical reactions. In a particular embodiment of the invention, the fluorophore is coupled to a cysteine from the polypeptide.

In a particular embodiment, the invention relates to a compound comprising a polypeptide and a fluorophore, said polypeptide being chosen in the group consisting of:
a) A polypeptide comprising the sequence SEQ ID N^{o} 12, the sequence SEQ ID N^{o} 14 and the sequence SEQ ID N^{o} 1,
b) A polypeptide comprising the sequence SEQ ID N^{o} 12, the sequence SEQ ID N^{o} 14 and the sequence SEQ ID N^{o} 2,
c) A polypeptide comprising the sequence SEQ ID N^{o} 12, the sequence SEQ ID N^{o} 14 and the sequence SEQ ID N^{o} 3,
d) A polypeptide comprising the sequence SEQ ID N^{o} 12, the sequence SEQ ID N^{o} 14 and the sequence SEQ ID N^{o} 4, and
e) A polypeptide comprising the sequence SEQ ID N^{o} 12, the sequence SEQ ID N^{o} 14 and the sequence SEQ ID N^{o} 5.

In a particular embodiment, the invention relates to a compound comprising a polypeptide and a fluorophore, said polypeptide having an amino-acid sequence chosen from the list consisting of: the sequence SEQ ID N^{o} 15, the sequence SEQ ID N^{o} 16, the sequence SEQ ID N^{o} 17, the sequence SEQ ID N^{o} 18, the sequence SEQ ID N^{o} 19, the sequence SEQ ID N^{o} 20, the sequence SEQ ID N^{o} 21, the sequence SEQ ID N^{o} 22, the sequence SEQ ID N^{o} 23, the sequence SEQ ID N^{o} 24, the sequence SEQ ID N^{o} 25, the sequence SEQ ID N^{o} 26, the sequence SEQ ID N^{o} 27, the sequence SEQ ID N^{o} 28, the sequence SEQ ID N^{o} 29.

In a particular embodiment, the invention relates to a compound comprising a polypeptide and a fluorophore, said polypeptide having an amino-acid sequence chosen from the list consisting of: the sequence SEQ ID N^{o} 31, the sequence SEQ ID N^{o} 32, the sequence SEQ ID N^{o} 33, the sequence SEQ ID N^{o} 34 and the sequence SEQ ID N^{o} 35.

In an even more particular embodiment, the invention relates to a compound comprising a polypeptide and a fluorophore, said polypeptide being chosen in the group consisting of:
A polypeptide comprising the sequence SEQ ID N^{o} 12, the sequence SEQ ID N^{o} 14 and the sequence SEQ ID N^{o} 1,
A polypeptide comprising the sequence SEQ ID N^{o} 12, the sequence SEQ ID N^{o} 14 and the sequence SEQ ID N^{o} 2,
A polypeptide comprising the sequence SEQ ID N^{o} 12, the sequence SEQ ID N^{o} 14 and the sequence SEQ ID N^{o} 3,
A polypeptide comprising the sequence SEQ ID N^{o} 12, the sequence SEQ ID N^{o} 14 and the sequence SEQ ID N^{o} 4, and
A polypeptide comprising the sequence SEQ ID N^{o} 12, the sequence SEQ ID N^{o} 14 and the sequence SEQ ID N^{o} 5,
   wherein the fluorophore is Cy3 and is coupled to a cysteine residue of said polypeptide.

In an even more particular embodiment, said polypeptide has an amino-acid sequence chosen from the list consisting of: the sequence SEQ ID N^{o} 15, the sequence SEQ ID N^{o} 16, the sequence SEQ ID N^{o} 17, the sequence SEQ ID N^{o} 18, the sequence SEQ ID N^{o} 19, the sequence SEQ ID N^{o} 20, the sequence SEQ ID N^{o} 21, the sequence SEQ ID N^{o} 22, the sequence SEQ ID N^{o} 23, the sequence SEQ ID N^{o} 24, the sequence SEQ ID N^{o} 25, the sequence SEQ ID N^{o} 26, the sequence SEQ ID N^{o} 27, the sequence SEQ ID N^{o} 28, the sequence SEQ ID N^{o} 29, the fluorophore is Cy3 and is coupled to a cysteine residue of said polypeptide.

In a more particular embodiment, said polypeptide has an amino-acid sequence chosen from the list consisting of: the sequence SEQ ID N^{o} 31, the sequence SEQ ID N^{o} 32, the sequence SEQ ID N^{o} 33, the sequence SEQ ID N^{o} 34, the sequence SEQ ID N^{o} 35, and wherein the fluorophore is Cy3 and is coupled to a cysteine residue of said polypeptide.

In another particular embodiment, the invention relates to a compound comprising a polypeptide and a fluorophore, said compound also comprising a cell-penetrating peptide sequence and/or a protein tag.

The compound of the invention may be prepared to allow its direct use in vitro, in cell extracts, in a cell, in a cell culture, including tissue culture, or on animal and/or human tissues, originating for example from biopsies. Thus, in an embodiment, the compound of the invention further comprises means to penetrate the cell membrane. In the list consisting of the means to penetrate cell membrane known by the skilled person, one can mention cell penetrating peptides. As used herein, the terms “cell penetrating peptide”, “cell-permeable peptides”, “protein-transduction domains (PTD)”, “membrane-translocation sequences (MTS)”, are equivalent. As used herein, the term “cell penetrating peptide” (CPP) refers to a short polycationic or amphiphilic peptide, for example comprising 5 to 40 amino acid, which can readily cross biological membranes and capable of facilitating cellular uptake of various molecular cargos, in vitro and/or in vivo. As used herein, the term “molecular cargo” refers to a molecule in the list consisting of chemical molecules, peptides, polypeptides, proteins or nucleotides.

In a preferred embodiment, the compound of the invention further comprises a cell penetrating peptide (CPP) sequence. Preferably, the cell penetrating peptide according to the invention is capable of facilitating cellular uptake of peptides, polypeptides or proteins. More preferably, the cell penetrating peptide according to the invention is capable of facilitating cellular uptake of peptides of a more than 5 amino acids, a cell penetrating peptide is capable of facilitating cellular uptake of peptides of up to at least 500 kDa (Kurzawa et al. 2010; Morris et al. 2001).

Such cell penetrating peptides are well known by the skilled person, and are described thoroughly in Grdisa et al., 2011, or Matjaz et al., (2005), Morris et al. 2008, Fonseca et al., 2009, Heitz et al. 2009.

According to the invention, the cell penetrating peptide is associated with the compound of the invention through covalent bonds, or through non-covalent interactions.

In another embodiment, the compound of the invention may further comprise a protein tag. Protein tags are peptide sequences genetically grafted onto a recombinant protein and are often removable by chemical agents or by enzymatic means, such as proteolysis or protein splicing. Protein tags are attached to proteins for various purposes and are well known by the skilled person and may for example be chosen in the list consisting of Isopeptag, BCCP, Myc-tag, Calmodulin-tag, FLAG-tag, HA-tag, His-tag, Maltose binding protein-tag, Nus-tag, Glutathione-S-transferase-tag, Green Fluorescent Protein-tag, Red Fluorescent Protein tag and other genetically encoded autofluorescent proteins, Thioredoxin-tag, S-tag, Softag 1, Softag 3, Strep-tag, SBP-tag, Ty tag, V5 tag or TC tag.

In yet another embodiment of the invention, the compound of the invention may have such attributes as being non-hydrolyzable, thereby increasing the stability against proteases or other physiological conditions which degrade the corresponding peptide. For example, peptide analogs can be generated using benzodiazepines, substituted γ-lactam rings, C7 mimics, β-turn dipeptides cores, β-aminoalocohols, diaminoketones, and methylene amino-modified. Also, several surrogates of the amide bond, including in the group of trans-olefins, fluoroalkylene, methyleneamino, phosphonamides or sulfonamides can be used in order to increase the half-life of the polypeptide.

The compound of the invention may be obtained by standard methods known in the art, for example chemical synthesis, and is not limited to a particular method. For example, the compound may be obtained by recombinant protein engineering.

The invention also relates to compositions comprising said compound and a pharmaceutically acceptable carrier.

As used herein, the term “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency or listed in a generally recognized pharmacopeia for use in animals, and more particularly in humans.

As used herein, the term “carrier” refers to a diluent, adjuvant, excipient, or vehicle. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

The compound of the invention may be solubilized in a buffer or water or incorporated in emulsions and microemulsions. Suitable buffers include, but are not limited to, phosphate buffered saline Ca++/Mg++ free (PBS), phosphate buffered saline (PBS), normal saline (150 mM NaCl in water), Tris buffer and surfactants.

There are numerous causes of peptide instability or degradation, including hydrolysis and denaturation. Hydrophobic interaction may cause clumping of molecules together (i.e. aggregation). Thus, in an embodiment, the composition according to the invention further comprises stabilizers.

Stabilizers according to the invention include cyclodextrine and derivatives thereof (see for reference U.S. Pat. No. 5,730,969). Suitable preservatives such as sucrose, mannitol, sorbitol, trehalose, dextran and glycerin can also be added to stabilize the final formulation. A stabilizer selected from ionic and non-ionic surfactants, D-glucose, D-galactose, D-xylose, D-galacturonic acid, trehalose, dextrans, hydroxyethyl starches, and mixtures thereof may be added to the formulation. Addition of alkali metal salt or magnesium chloride may stabilize the compound according to the invention. The peptide may also be stabilized by contacting it with a saccharide selected from the group consisting of dextran, chondroitin sulphuric acid, starch, glycogen, dextrin, and alginic acid salt. Other sugars that can be added include monosaccharides, disaccharides, sugar alcohols, and mixtures thereof (E.g., glucose, mannose, galactose, fructose, sucrose, maltose, lactose, mannitol, xylitol). Polyols may stabilize a peptide, and are water-miscible or water-soluble. Suitable polyols may be polyhydroxy alcohols, monosaccharides and disaccharides including mannitol, glycrol, ethylene glycol, propylene glycol, trimethyl glycol, vinyl pyrrolidone, glucose, fructose, arabinose, mannose, maltose, sucrose, and polymers thereof. Various excipients may also stabilize peptides, including serum albumin, amino acids, heparin, fatty acids and phospholipids, surfactants, metals, polyols, reducing agents, metal chelating agents, polyvinyl pyrrolidone, hydrolysed gelatin, and ammonium sulfate.

The composition of the invention may be formulated according to standard pharmaceutical practice. The composition may be formulated in a form suitable for oral, enteral or parenteral administration, including the intravenous, intramuscular, intraperitoneal, subcutaneous, rectal, respiratory and topical routes of administration,

Ways to penetrate the cellular membrane may involve specific formulations of the compound according to the invention, preferentially formulations suitable for administration to cells or animal and/or human tissues. In particular, the compound of the invention may be encapsulated in liposomes to form pharmaceutical preparations suitable for administration to cells and animal and/or human tissues (see for reference U.S. Pat. No. 5,190,762).

Other types of lipid aggregates may be used to formulate the compound of the invention. Such aggregates include liposomes, unilamellar vesicles, multilamellar vesicles, micelles and the like, having particle sizes in the nanometer to micrometer range. Methods of making lipid aggregates are by now well-known in the art.

The invention also relates to the use of at least one compound and/or a composition of the invention for fluorescence imaging. In a particular embodiment, the invention also relates to the use of at least one compound and/or a composition of the invention for fluorescence medical imaging, preferably endoscopic imaging. In another embodiment, the invention also relates to the use of at least one compound and/or a composition of the invention for in vitro fluorescence imaging.

The invention also relates to a method for medical imaging, especially endoscopic imaging, comprising the steps of:
a) administering to a subject a compound of the invention,
b) illuminating at least one part of an organ of said subject at a wavelength corresponding to the wavelength of the fluorophore of the compound of the invention, and
c) obtaining an image with an apparatus detecting the fluorescence emitted by said administered compound.

The skilled person may select the appropriate imaging apparatus depending on the fluorophore of the compound according to the invention.

According to the present invention, the term “effective amount” of a composition means the amount which is sufficient to allow for measurement of the fluorescence in the subject, particularly. It is understood that the effective dosage will be dependent upon the age, sex, health, and weight of the recipient, the nature of the disease or condition being investigated, and the nature of the effect desired. The effective amount can be tailored to the individual subject, as is understood and determinable by one of skill in the art, without undue experimentation.

The invention furthermore relates to methods for determining if at least one CDK and/or one Cyclin-CDK complex is active, comprising the steps of:
a) providing at least one compound according to the invention,
b) contacting said compound with said CDK and/or Cyclin-CDK complex,
c) illuminating said compound and said CDK and/or Cyclin-CDK complex with an excitation light,
d) determining the fluorescence signal emitted by said compound,
e) comparing said fluorescence signal with a reference fluorescence signal, and
f) determining from the comparison of step e) if at least one CDK and/or one Cyclin-dependent-CDK is active.

According to the invention, contacting the compound of the invention with CDK and/or Cyclin-CDK complex is performed, for example, by contacting the compound of the invention directly with a recombinant form of CDK/Cyclin or with solutions, extracts, particularly cell extracts, living cells, preferably living cells in an in vitro culture, animal tissues, preferably animal tissues in an in vitro culture, or any type of sample containing CDK and/or Cyclin-CDK complex whose activity is to be determined.

The skilled person will easily adapt the intensity and wavelength of the excitation light of step c), for example depending on the fluorophore of the compound according to the invention of step a). In an embodiment, the illumination of step c) is performed at a wavelength corresponding to the excitation wavelength of the fluorophore of the compound according to the invention of step a). Various light sources may be used to provide for the excitation light, including lasers, photodiodes, and lamps, preferably xenon arcs lamps and mercury-vapor lamps.

According to the invention, determining the fluorescence in step d) can be achieved by any technique and using any appropriate apparatus known in the art. Any fluorimeter such microscope, fluorescence-activated cell sorter (FACS) or other device adapted to measure the properties of emitted light, preferably fluorescence light may be used to determine the fluorescence of step d).

As used herein, “determining the fluorescence signal emitted” refers to measuring the properties of the emitted fluorescence, such as for example measuring the wavelength spectrum, intensity or half-life of the emitted fluorescence. In an embodiment, determining the fluorescence emitted is achieved by measuring the wavelength spectrum of the emitted fluorescence. In another embodiment, determining the fluorescence emitted is achieved by measuring the intensity of the emitted fluorescence at a given wavelength (generally its maximum).

According to the invention, “comparing the fluorescence signal” means comparing the properties of the emitted fluorescence signal and the properties of the reference fluorescence signal. In an embodiment, comparing the fluorescence signal means comparing the wavelength spectrum of the emitted fluorescence and the wavelength spectrum of the fluorescence reference. In another embodiment, comparing the fluorescence signal means comparing the intensity of the emitted fluorescence to the intensity of the fluorescence reference at a chosen wavelength.

According to the invention, the “reference fluorescence signal” is a predetermined measure of fluorescence, obtained from a biological sample with a known CDK and/or Cyclin-CDK complex activity. In an embodiment, the reference fluorescence signal is a predetermined measure of fluorescence obtained from a reference biological sample wherein said CDK and/or Cyclin-CDK complex is known to be active. In another embodiment, the reference fluorescence signal is a predetermined measure of fluorescence obtained from a reference biological sample wherein said CDK and/or Cyclin-CDK complex is known to be inactive. Activity may be determined by conventional techniques known by the skilled person.

The compound according to the invention emits a fluorescence that changes, for example in intensity or wavelength, depending on the conformation of the compound and inherently on the phosphorylation status of serine S₂ residue and/or threonine T residue of said X₁-S₁-S₂/T-P-X₂ sequence of said substrate domain. Accordingly, the fluorescence emitted by the fluorophore of the invention may change if the CDK and/or Cyclin-CDK complex of interest changes its activity, for example following the action of an inhibitor of said CDK and/or Cyclin-CDK complex of interest. Thus, a compound according to the invention may be useful for example for in vitro in real-time studies, as well as for screening, particularly high throughput screening.

The invention thus relates to methods for determining the kinetics of at least one CDK and/or Cyclin-CDK complex activity, comprising the steps of:

a) providing at least one compound according to the invention,

b) contacting said compound with said CDK and/or Cyclin-CDK complex,

c) illuminating said CDK and/or Cyclin-CDK complex and compound with an excitation light,

d) determining fluorescence signal emitted by said compound,

e) repeating steps c) and d) at different times, at least once,

f) comparing said fluorescence of step d) and step e), and

g) determining the kinetics of at least one CDK and/or Cyclin-CDK complex activity from the comparison of step f).

The invention also relates to methods for determining if a molecule is a modulator of the activity of at least one CDK and/or Cyclin-CDK complex, which are methods for screening a plurality of products for their ability to modulate the activity of at least one CDK and/or Cyclin-CDK complex, comprising the steps of:

a) providing at least one compound according to the invention,

b) contacting said compound with said CDK and/or Cyclin-CDK complex,

c) illuminating said CDK and/or Cyclin-CDK complex and compound with an excitation light,

d) determining the fluorescence signal emitted by said compound,

e) comparing said fluorescence signal with a reference fluorescence signal, and

f) determining from the comparison of step e) if said at least one compound is a modulator of the activity of at least one CDK and/or Cyclin-CDK complex.

According to the invention, the modulator is an activator or an inhibitor of the activity of at least one CDK and/or Cyclin-CDK complex. Preferably, the modulator is an inhibitor of the activity of at least one CDK and/or Cyclin-CDK complex.

The invention also relates to methods for the in-vitro diagnosis of CDK and/or Cyclin-CDK complex hyperactivation in a subject, comprising the steps of:
a) providing at least one compound according to the invention,
b) contacting said compound with a biological sample from said subject,
c) illuminating said biological sample and compound with an excitation light,
d) determining fluorescence emitted by said compound,
e) comparing said fluorescence with a fluorescence reference, and
f) determining from the comparison of step d) if at least one Cyclin-dependent kinase is hyperactive.

As used herein, the term “CDK and/or Cyclin-CDK complex hyperactivation” means that at least one CDK and/or Cyclin-CDK complex is hyperactive, e.g. shows an above normal activity. According to the invention, the above normal activity is determined by comparison of the test value with a reference value. The reference value according to the invention is for example a value obtained by the present method with a biological sample wherein the CDK and/or Cyclin-CDK complex activity is normal, such as for example non transformed cell lines, for example normal diploid fibroblast, preferably the HS68 cell line (ATCC code HTB-138). An example of the determination of such hyperactivation is shown in FIG. 6B of the present Application, wherein the activity in Hela cellular extracts is compared to the activity in HS68 extracts.

The terms “individual”, “subject”, “host” and “subject” are used herein interchangeably and refer to any subject for whom diagnosis is desired, particularly humans. Other subjects may include cattle, dogs, cats, guinea pigs, rabbits, rats, mice, horses, and the like. In some preferred embodiments the subject is a human.

As used herein, the term “biological sample” refers to biological material from a subject. The sample assayed by the present invention is not limited to any particular type. Samples include, as non-limiting examples, single cells, multiple cells, tissues, tumors, biological fluids, biological molecules, or supernatants and/or extracts of any of the foregoing. Examples include tissue removed for biopsy, tissue removed during resection, blood, serum, plasma, sputum, urine, lymph tissue, lymph fluid, cerebrospinal fluid, mucous, skin, saliva, gastric secretions, semen, seminal fluid, tears, spinal tissue or fluid, cerebral fluid, trigeminal ganglion sample, a sacral ganglion sample, adipose tissue, lymphoid tissue, placental tissue, upper reproductive tract tissue, gastrointestinal tract tissue, male genital tissue and fetal central nervous system tissue and stool samples.

The sample used will vary based on the assay format, the detection method and the nature of the tissues, cells or extracts to be assayed. Methods for preparing samples are well known in the art and can be readily adapted in order to obtain a sample that is compatible with the method utilized.

According to the invention, the reference fluorescence is a predetermined measure of fluorescence, obtained from a biological sample where the CDK and/or Cyclin-CDK complex of interest is known to be normally active. In an embodiment, the reference fluorescence is a predetermined measure of fluorescence obtained from a reference biological sample from the subject according to the invention, wherein the reference biological sample is known to have CDK and/or Cyclin-CDK complex of interest normally active. Preferably, the reference fluorescence is a predetermined measure of fluorescence obtained from a biological sample from a subject known to have CDK and/or Cyclin-CDK complex of interest normally active.

The disregulation of Cyclin-dependant kinase level or activation is suspected to contribute to the observed sustained aberrant proliferation of cancer cells, and as such is considered a hallmark of several diseases. Indeed, the levels of either Cyclin of Cyclin-dependent kinases, as well as the kinase activity of Cyclin-dependent kinases are frequently altered in human cancers. A Cyclin-dependant kinase hyperactivation has been reported in a wide range of cancers including breast, ovarian, prostate, colorectal, and lung cancers, as well as lymphoma, myeloma, sarcoma, and glioblastoma

The invention thus discloses a method for the in vitro diagnosis of cancer in a subject, comprising the steps of:

a) providing at least one compound according to the invention,

b) contacting said compound with a biological sample from said subject,

c) illuminating said biological sample and compound,

d) determining the fluorescence emitted by said compound,

e) comparing said fluorescence with a fluorescence threshold reference, and

f) diagnosing a cancer in said subject from the comparison of step e) if the fluorescence of step d) is above the fluorescence threshold reference.

As used herein, “cancer” refers to primary or metastatic cancers, leukemia, or lymphomas, colon cancer, liver cancer, testicular cancer, thymus cancer, breast cancer, skin cancer, esophageal cancer, pancreatic cancer, prostatic cancer, uterine cancer, cervical cancer, lung cancer, bladder cancer, ovarian cancer, multiple myeloma, melanoma and glioblastoma. Preferably, the cancer according to the invention is a CDK and/or Cyclin-CDK complex-associated cancer. By “CDK and/or Cyclin-CDK complex associated cancer” it is herein referred to cancers associated with an hyperactivity of at least one CDK and/or Cyclin-CDK complex.

In an embodiment, the fluorescence threshold reference is a predetermined measure of fluorescence, obtained from one or several biological sample from subjects known to have cancer. In a particular embodiment, the fluorescence threshold reference is a statistically relevant data obtained from predetermined measures of fluorescence, obtained several biological samples from subjects known to have cancer.

The invention also discloses a method for evaluating in vitro the therapeutic efficiency of a cancer treatment for a subject, comprising the steps of:

a) providing at least one compound according to the invention,

b) contacting said compound with a biological sample from said subject,

c) illuminating said biological sample and compound,

d) determining the fluorescence emitted by said compound,

e) comparing said fluorescence of step d) with a fluorescence determined in said subject before the treatment, and

f) determining that said treatment is therapeutically efficient for said subject from the comparison of step e) if the fluorescence of step d) is lower than the fluorescence determined in said subject before the treatment.

The following examples are provided herein for purposes of illustration only and are not intended to be limiting unless otherwise specified.

### BRIEF DESCRIPTION OF THE DRAWINGS

FIGS. 1A to 1C: Design and purification of CDKACTH1

FIG. 1A: Schematic representation of CDKACT, the substrate sequence bears a phosphorylation site and a unique cysteine two residues upstream. For CDKACTH1, the substrate sequence derives from Histone H1. Phosphorylation of CDKACT biosensors promotes fluorescence enhancement of the fluorescent probe coupled to the unique cysteine, associated with the conformational change upon interaction of the phosphobinding domain with the phosphorylated substrate.

FIG. 1B. Purification on SDS-PAGE and labelling of GST-CDKACTH1

FIG. 1C. Radioactive endpoint assay: the products of the phosphorylation of Histone H1 (control) or CDKACTH1 by recombinant CDK1/Cyclin B or recombinant CDK2/Cyclin A (phosphorylated by recombinant CIV) are shown. The radioactive assay was performed with 100 nM kinase and 50 uM H1 substrate.

FIGS. 2A to 2D: Fluorescent real-time assay of CDK2/Cyclin A activity using CDKACTH1

FIG. 2A. Fluorescence kinase assay of 25 nM CDKACTH1-Cy3 upon incubation with 25 nM recombinant activeCDK2/Cyclin A with or without ATP/MgCl2—or without kinase

FIG. 2B. Fluorescence kinase assay of 25 nM CDKACTH1-Cy3 incubated with 25 nM recombinant active CDK2/Cyclin A with or without 20 uM roscovitine kinase inhibitor

FIG. 2C. Competition Assay: Fluorescence of 25 nM CDKACTH1-Cy3 incubated with 25 nM recombinant active CDK2/Cyclin A supplemented with 25 nM or 100 nM unlabelled CDKACTH1 or 25 nM or 500 nM histone

FIG. 2D. Fluorescence kinase assay of 25 nM CDKACTH1-Cy3 upon incubation with 25 nM recombinant active CDK2/Cyclin A with or without ATP/MgCl2, and of the CDKACTH1 phosphorylation site mutant (“MutantCy3”) upon incubation with 25 nM recombinant active CDK2/Cyclin A.

In FIG. 2A-C, the relative fluorescence of CDKACTH1-Cy3 is expressed as a function of the reaction time. In FIG. 2D, the percentage of relative fluorescence of CDKACTH1-Cy3 and of CDKACTH1 mutant is expressed as a function of the reaction time.

Real-time fluorescence assays were performed at 30° C. in 96-well plates. Changes in CDKACT-Cy3 fluorescence were monitored at 590 nm following excitation at 544 nm and were substracted from background fluorescence of the sensor alone. In each graph phosphorylation by CDK2/Cyclin A/CIV are shown for comparison.

FIGS. 3A to 3E: In vitro characterization of CDKACTH1 specificity

FIG. 3A: Kinase activity of 100 nM recombinant CDK1/CyclinB incubated with 10 nM CDKACTH1-Cy3 with or without ATP/MgCl2, 20 uM roscovitine or 20 uM RO3306

FIG. 3B: Kinase activity of 75 nM recombinant CDK4/CyclinD incubated with 25 nM CDKACTH1-Cy3 with or without 20 uM roscovitine or 20 uM PD-0332991 inhibitor

FIG. 3C: Kinase activity of 25 nM of recombinant CDKACTH1 incubated with 10, 25, 50 or 75 nM of CIV kinase. FIG. 3D: Kinase activity of 25 nM of recombinant CDKACTH1 incubated with 25, 50, 75 nM Plk1 kinase. FIG. 3E: Kinase activity of 25 nM of recombinant CDKACTH1 incubated with 25, 50, 75 nM Plk3 kinase. In graphs 3C, 3D and 3E, phosphorylation by CDK2/cyclin A and by kinase CIV are shown for comparison.

FIGS. 4A to 4C: Application of CDKACTH1 to monitor kinase activity in mammalian cell extracts

FIGS. 4A-C show the phosphorylation of CDKACTH1 by 20 ug HeLa cell extracts supplemented with increasing concentrations of CDK/Cyclin inhibitors, said inhibitors being respectively: A. Roscovitine B. RO3306 C. PD-0332991 inhibitor

Real-time fluorescence assays were performed at 30° C. in 96-well plates. Changes in CDKACT-Cy3 fluorescence were monitored at 590 nm following excitation at 544 nm and were substracted from background fluorescence of the sensor alone.

FIGS. 5A and 5B: Profiling activity of CDK/Cyclin complexes enriched from mammalian cell extracts by size exclusion chromatography

FIG. 5A: Gel Filtration Profile of HeLa cells extracts injected onto a Superose 12 FPLC column and Western blot analysis of the fractions (A7 to B10) eluted from the size exclusion chromatography. FIG. 5B: CDKACTH1-Cy3 fluorescence assay, CDKACT-Cy3 was incubated with the different HeLa fractions (A7 to B8). Real-time fluorescence assays were performed at 30° C. in 96-well plates. Changes in CDKACT-Cy3 fluorescence were monitored at 590 nm following excitation at 544 nm and were substracted from background fluorescence of the sensor alone.

FIGS. 6A to 6C: Application of CDKACTH1 to monitor kinase activity in mammalian cell extracts

FIG. 6A. CDKACTH1-Cy3 fluorescence corresponding to kinase activity of CDK/Cyclins in 40 ug HT2-19 cell extracts expressing or not CDK1 (ie in the presence or not of 50 uM IPTG). The inset shows the Western blot corresponding to the levels of CDK1. FIG. 6B. CDKACTH1-Cy3 fluorescence corresponding to kinase activity of CDK/Cyclins in 20 ug HeLa and HS68 cell extracts. The inset shows the Western blot corresponding to the levels of different CDKs and Cyclins in normalized cell extracts. FIG. 6C. CDKACTH1-Cy3 fluorescence corresponding to kinase activity of CDK/Cyclins in 20 ug cell extracts of, respectively, HeLa, A549, U20S and MCF7 cells. The average and standard deviation for three different experiments is shown. Real-time fluorescence assays were performed at 30° C. in 96-well plates. Changes in 25 nM CDKACT-Cy3 fluorescence in the presence of cell extracts were monitored at 590 nm following excitation at 544 nm and were substracted from background fluorescence of the sensor alone.

FIGS. 7A to 7E: Application of RFP-CDKACTH1-Cy5 to monitor kinase activity in living cells

FIG. 7A: Schematic representation of RFP-CDKACTH1, showing the RFP domain, the phosphate recognition domain and the substrate domain. FIG. 7B: RFP and Cy5 fluorescence of the RFP-CDKACTH1-Cy5 construct following internalization into living Hela cells. The kinase activity profile is shown for a representative field of 10 asynchronous, cycling cells. Fluorescence was acquired through time-lapse imaging on an inverted epifluorescence microscope. FIG. 7C: Representative example of the ratiometric quantification of Cy5/RFP fluorescence over time in a single dividing HeLa cell, following internalization of the RFP-CDKACTH1-Cy5 construct. Fluorescence acquired through time-lapse imaging on an inverted microscope. FIG. 7D: Ratiometric quantification of Cy5/RFP fluorescence in single non-dividing and dividing HeLa cells following internalization of RFP-CDKACTH1-Cy5. FIG. 7E: Ratiometric quantification of Cy5/RFP fluorescence in single non-dividing and dividing HeLa cells following internalization of the non-phosphorylatable mutant of RFP-CDKACTH1-Cy5

FIGS. 8A-C: CDKACTH1-Cy3/CDK2/Cyclin A close-dependency

FIGS. 8A-C show different concentrations of CDKACTH1-Cy3 incubated with 25, 50, 75, 100, 200 or 400 nM of recombinant CDK2/Cyclin A (phosphorylated by CIV).

FIGS. 9A-D: CDKACTH1-Cy3/CDK1/Cyclin B activity

FIGS. 9A-D show different concentrations of CDKACTH1-Cy3 incubated with 25, 50, 75, 100 nM of recombinant CDK1/Cyclin B (phosphorylated by CIV).

FIGS. 10A-10B: Inhibition of Phosphatases in cell extracts increases the fluorescence of CDKACTH1-Cy3 associated with kinase activity

CDKACTH1-Cy3 fluorescence was monitored over time in HeLa cells in the presence or absence of phosphatases inhibitors. The average and standard deviation for three different experiments is shown.

FIG. 11: Fluorescent real-time assay of CDK2/Cyclin A activity using CDKACT-WWH1-Cy5. The relative fluorescence of CDKACT-WWH1-Cy5 is expressed as a function of the reaction time. Real-time fluorescence assays were performed at 30° C. in 96-well plates. Assays were performed in the presence of 25 nM CDKACT-WWH1-Cy5 upon incubation with 25 nM recombinant activeCDK2/Cyclin A with ATP/MgCl2 (black dots), in the presence of kinase inhibitors: 20 microM Roscovitine (medium dots) or 20 microM PD0332991 (white dots). Changes in CDKACT-Cy5 fluorescence were monitored over time at 675 nm following excitation at 645 nm and background fluorescence of the sensor alone was substracted from the fluorescent signal of CDKACT-Cy5 in the presence of kinase.

FIGS. 12A and 12B: Fluorescent real-time assay of CDK2/Cyclin A activity in the presence of CDKACT-WWH1-Cy3 (FIG. 12A) or CDKACT-WWH1-Cy5 (FIG. 12B). The relative fluorescence of Cy3-CDKACTH1 and of Cy3-CDKACT-WWH1 is expressed as a function of the reaction time. Real-time fluorescence assays were performed at 30° C. in 96-well plates. Fluorescence emission of Cy3 was measured at 590 nm following excitation at 544 nm on Polarstar™ fluorimeter. FIG. 12A: Assays were performed in 200 ul, in the presence of 5 mM ATP& 20 M MgCl2, with 25 nM CDKACT-H1-Cy3 (black dots) or 25 nM of mutant CDKACT-H1-Cy3 (black squares), or in the absence of ATP/MgCl2, with 25 nM CDKACT-H1-Cy3 (triangles), or in the presence of CDKACT-H1-Cy3 and CDK2/CIV (stars). FIG. 12B: Assays were performed in the presence of ATP/MgCl2 and in the presence of 25 nM CDKACT-WWH1-Cy3 (black dots) or of mutant CDKACT-WWH1-Cy3 (medium dots), or in the absence of ATP/MgCl2 and in the presence of CDKACT-WWH1-Cy5 (white dots).

FIGS. 13A and 13B: In vivo use of the biosensor CDKACT-H1-Cy5

FIG. 13A: A375 xenograft tumoral volume is represented as a function of days post-implantation, whereas treatment was implemented with either Roscovitine (clear triangles) or DMSO vehicle control (squares), non-treated control group is represented (diamonds). FIG. 13B: the relative fluorescence of CDKACTH1-Cy5 is expressed in this histogram as a function of the tumour group, with, from left to right, DMSO vehicle treated-tumors, non-treated tumors and roscovitine treated tumors.

### EXAMPLES

### Example 1

### Design and Engineering of GST-CDKACTH1 and RFP-CDKACTH1

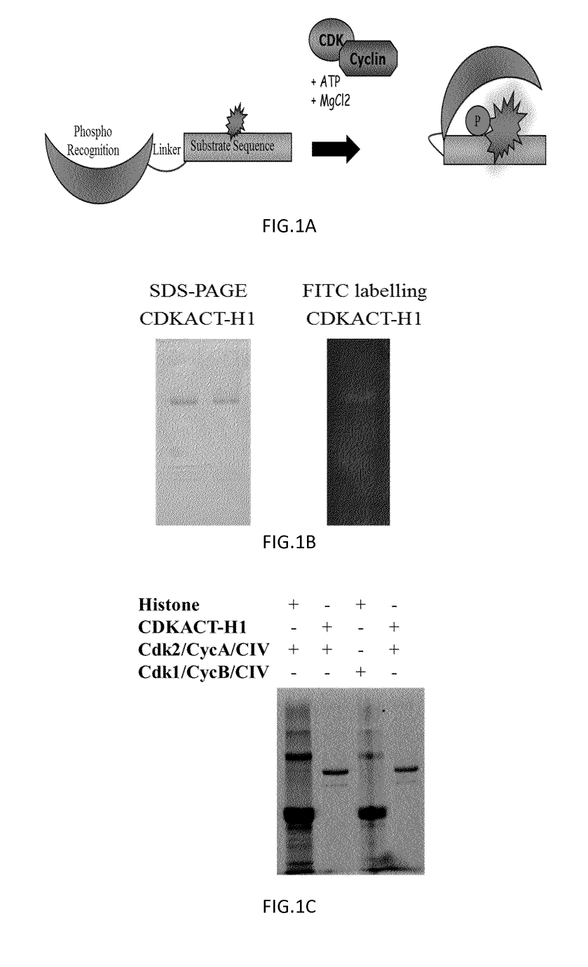

The CDKACT-H1 biosensor was engineered by cloning residues 367-603 of PBD from Plk1 into the pGex6P1 vector (BamHI/EcoRI) and mutagenizing all 6 cysteines into serines. A CDK substrate sequence derived from histone H1, bearing a TP phosphorylation site and a unique cysteine at position −2, GGCSTPKKAKKL was cloned downstream of the PBD, following a 12mer proline and glycine-rich linker providing flexibility between the PBD and the substrate sequence, PGAGGTGGLPGG. FIG. 1A shows a schematic representation of CDKACT-H1 and the principle of CDK/Cyclin activity detection. GST-CDKACTH1 was expressed in E. coli, then purified on High performance glutathione Sepharose in 50 mM TrisHCl, pH 7.4, 150 mM NaCl, then further purified by Gel Filtration chromatography. Note that the GST tag was necessary to preserve solubility and function of the biosensor, since its removal lead to complete precipitation of the biosensor. Following purification, CDKACTH1 was labelled with a 10fold molar excess Cy3-maleimide and further purified on NAPS columns to remove excess label.

RFP-CDKACT was engineered by cloning CDKACT into the pRSETB-mRFP vector, then expressed in E. coli and purified by HisTrap and gel filtration chromatography. RFP-CDKACTH1 was labeled with 10fold molar excess Cy5-maleimide and further purified on NAPS columns to remove excess label.

### Example 2

### In Vitro Characterization of CDKACT-H1 Biosensor

In order to characterize CDKACT-H1 biosensor in vitro, we first asked whether it could be used to monitor recombinant kinase activity in a real-time assay. CDKACT-H1 is phosphorylated by GST-CDK2/Cyclin A and GST-CDK1-Cyclin B in a standard kinase assay, using radioactive gamma-ATP and histone H1 as a positive control, following 30 min incubation at 30° C. A kinase assay was performed by addition of GST-CDK2/CIV complexed with His-Cyclin A to CDKACTH1, in the presence of 0.5 mM ATP and 5 mM MgCl2, and CDKACTH1 fluorescence was monitored over time.

### Material:

Protein expression and purification of Cyclin-dependent Kinases: Recombinant CDKs and cyclins were expressed in E. coli by IPTG induction and purified by chromatography as described previously (Heitz et al., Biochemistry. 36, 4995-5003 (1997)). GST-CDK2/CIV CyclinA and GST-CDK1/CIV Cyclin B were co-purified on a single column so as to elute complexes, the activity of which was verified in a radioactive endpoint assay, as described below.

### Radioactive Kinase Assays:

Phosphorylation of 5 uM CDKACT-H1 or histone H1 were performed in 50 mM Tris pH 7.5, 5 mM MgCl2, 0.5 mM ATP, and 33P-gamma-ATP for 30 min at 30° C. The reaction was stopped by addition of Laemmli buffer, and samples were run on SDS-PAGE then exposed by autoradiography.

### Real-Time Fluorescence Experiments:

Fluorescence kinase assays were performed in 96-well plates in a thermostated chamber (Polarstar BMG) at 30° C. in 200 ul phosphate buffer saline, pH 7.2, 150 mM NaCl, in the presence of 5 mM MgCl2, 0.5 mM ATP, except when stated otherwise. The fluorescence of Cy3-labelled CDKACTH1 with recombinant CDK/Cyclin complexes or with cell extracts was monitored over time. Changes in Cy3 fluorescence emission were recorded at 590 nm following excitation at 544 nm. In all experiments, the fluorescence of CDKACTH1-Cy3 alone was substracted from all other values. Data analysis and curve fitting were performed using the GraFit Software (Erathicus Ltd).

### Inhibitors:

Roscovitine (stock 1 mg/ml or 2.8 mM) and the PD-0332991 (stock 5 mM) inhibitor were purchased from Euromedex. RO3306 was purchased from CalbioChem. All stock solutions were made in DMSO.

### Antibodies for Western Blotting and Indirect Immunofluorescence:

Antibodies against Cyclin A (H432, sc-751), Cyclin B1 (GNS1, sc-245), Cyclin D1 (C20, sc-717), Cdk1 (C19, sc-954) Cdk2 (M2, sc-163), and Cdk4 (C22, sc-260) were purchased from Tebu-Bio (Santa-Cruz), anti-actin from Sigma (A2668), and used at 1:1000 dilution for Western blotting, except for anti-cyclin B1 used at 1:500 dilution.

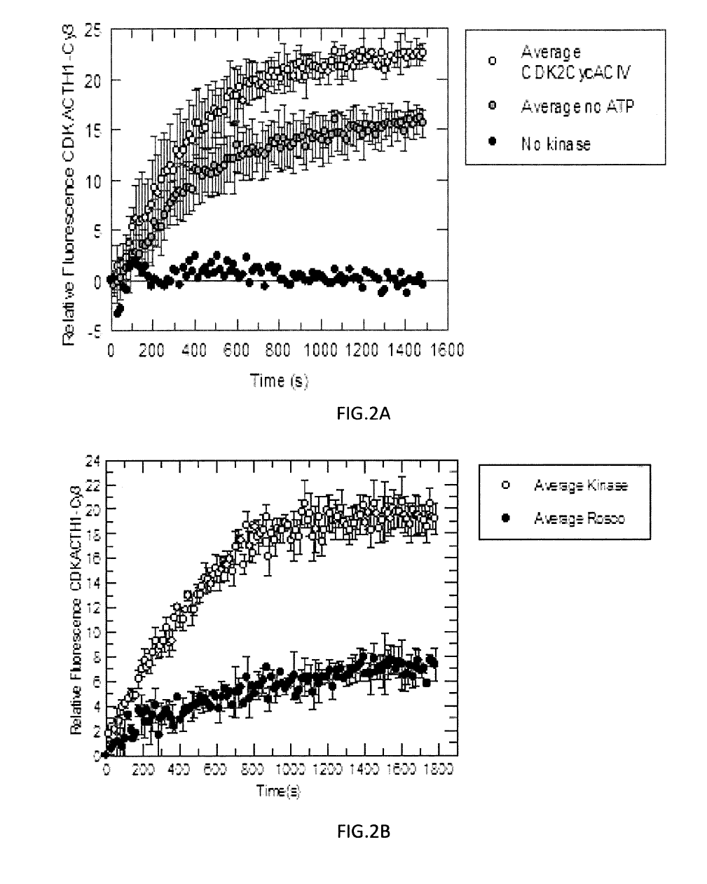

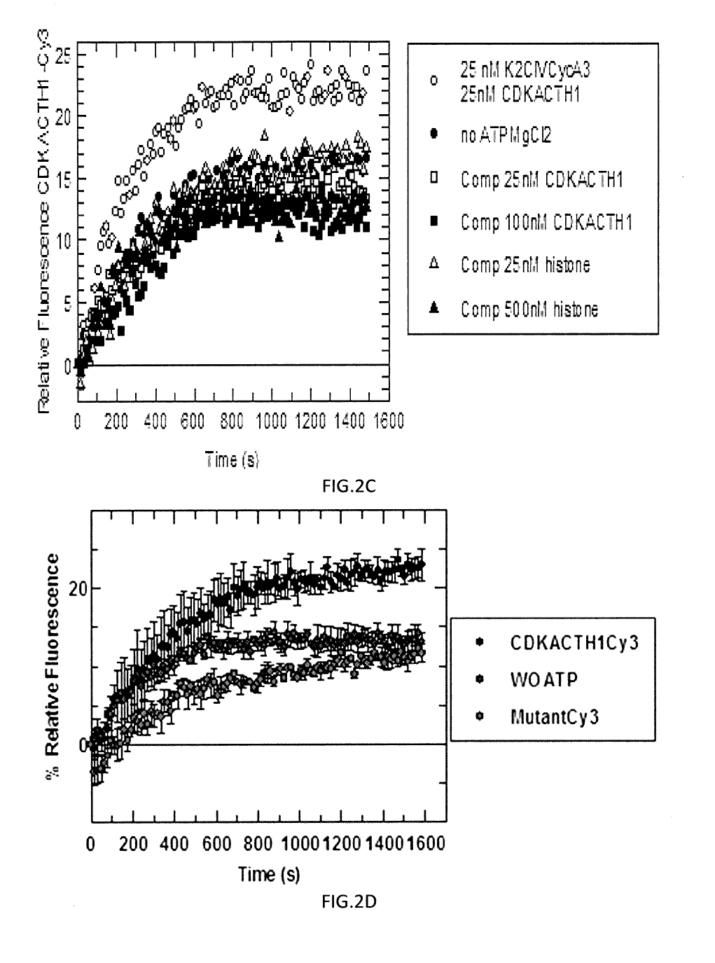

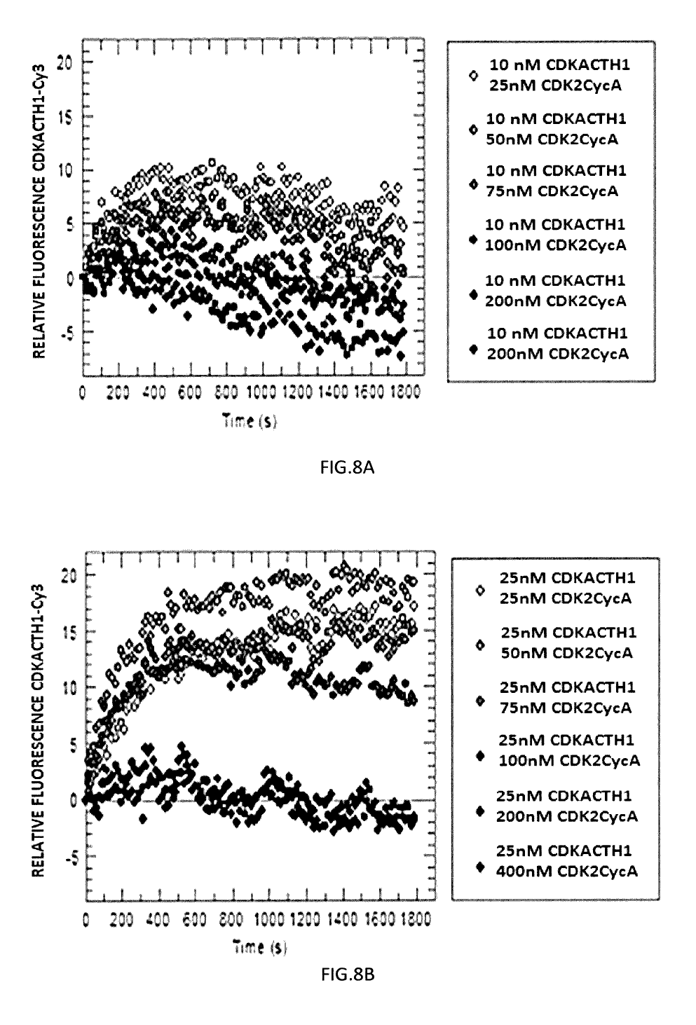

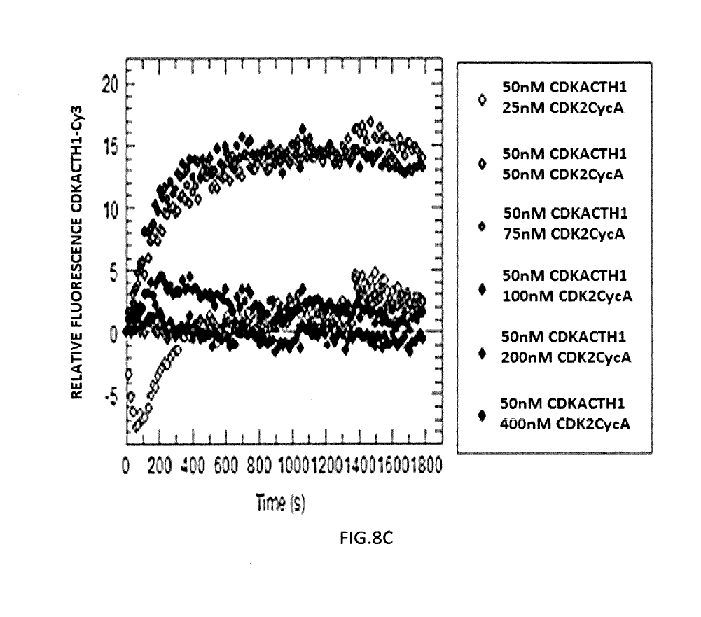

In order to determine the best working conditions, we first analyzed the dose-dependency of different fixed concentrations of CDKACTH1 for different concentrations of kinase (FIG. 2A, FIGS. 8A-C). In these experiments, we found that the best response was obtained at a concentration of 25 nM CDKACTH1 for 25-50 nM CDK2/CyclinA. We therefore chose these conditions to further examine the fluorescence profile in the presence or absence of MgCl2/ATP. These profiles revealed clearly that in the absence of MgCl2/ATP still induced a partial increase in the fluorescence of CDKACTH1, indicative of its binding to the sensor. They also showed that in the absence of cyclinA, the fluorescent signal first decreased and then slowly returned to a basal level, inferring that monomeric CDK2 cannot bind the sensor properly in the absence of cyclinA, as has already been reported for many CDK/Cyclin substrates. To further validate that the increase in CDKACTH1 fluorescence was due to kinase activity, we added 20 uM roscovitine, a broad inhibitor of CDK/Cyclins or RO3306, which preferentially inhibits CDK1. Whereas roscovitine inhibited the fluorescence of CDKACTH1 almost completely, RO3306 had little effect at this concentration (FIG. 2B). Finally, we asked whether this sensor behaved as a competitive substrate with unlabelled sensor, or with histone H1 (FIG. 2C). This experiment showed that histone and unlabelled sensor prevented full phosphorylation of CDKACTH1.

In order to characterize CDKACT-H1 biosensor in vitro, we first asked whether it could be used to monitor recombinant kinase activity in a real-time assay. A kinase assay was performed by addition of GST-CDK2/CIV complexed with His-Cyclin A to CDKACTH1, in the presence of 0.5 mM ATP and 5 mM MgCl2, and CDKACTH1 fluorescence was monitored over time.

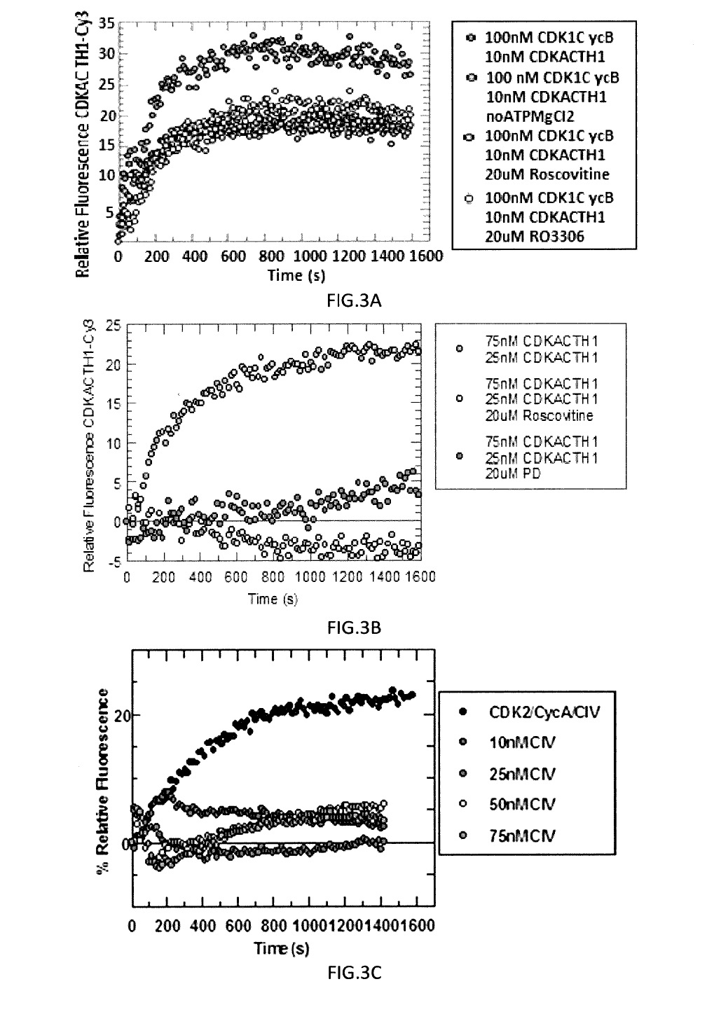

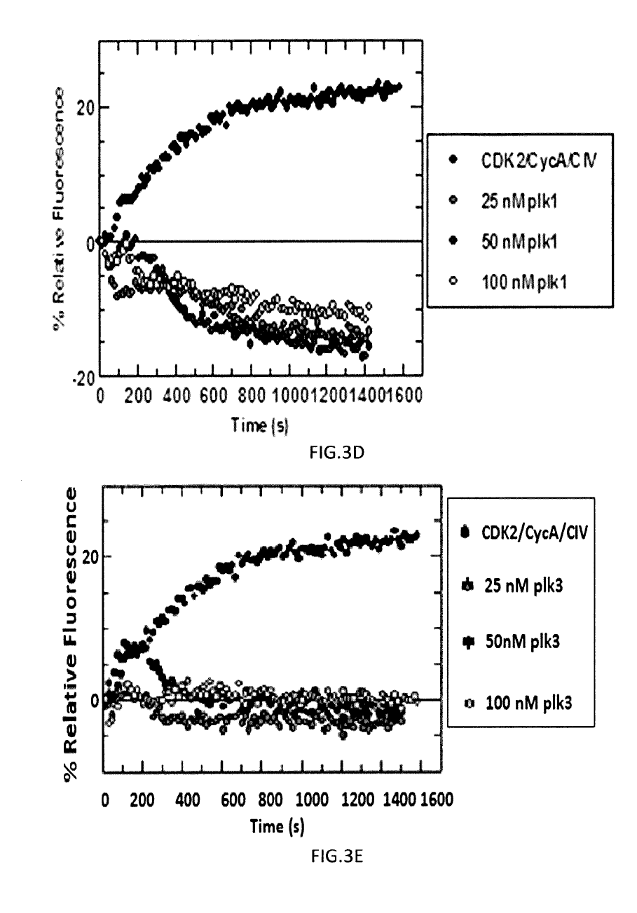

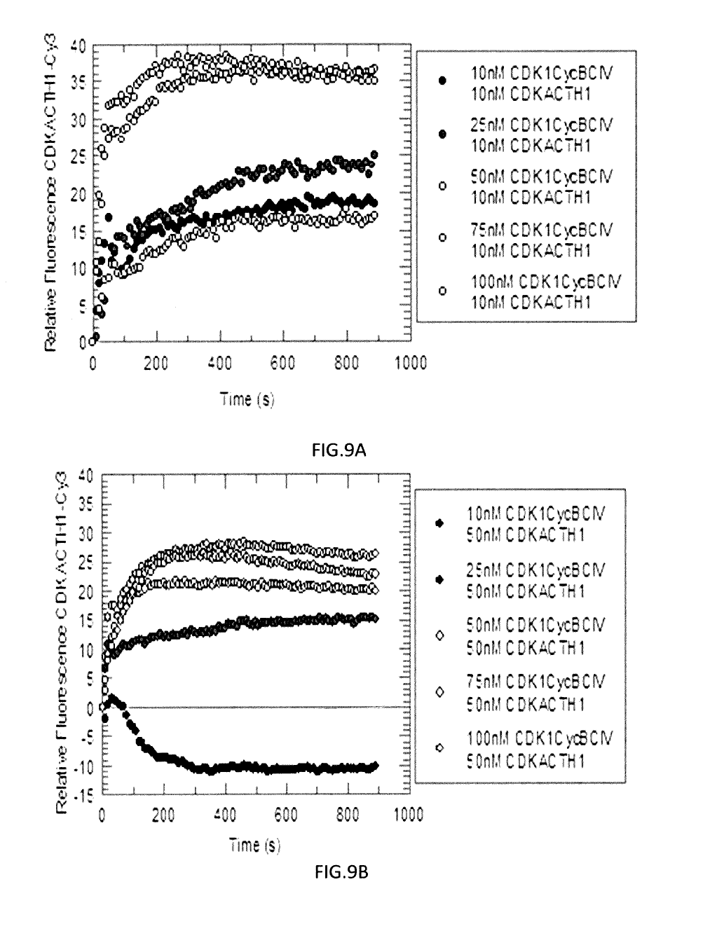

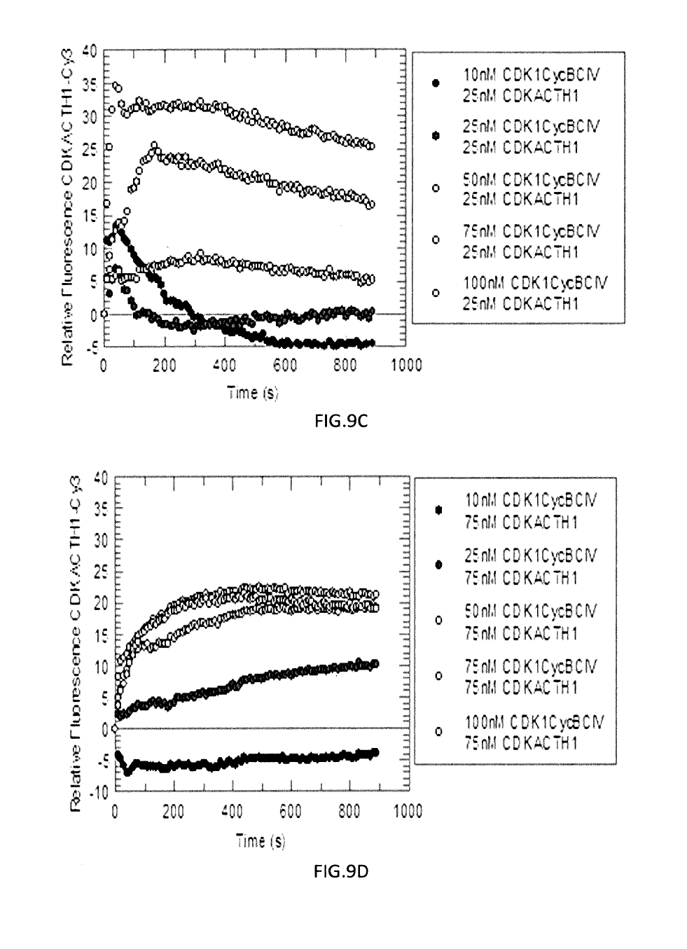

To address the issue of specificity, we asked whether other kinases could phosphorylate and thereby affect the fluorescence of CDKACTH1. We first examined CDK1/Cyclin B and CDK4/Cyclin D (FIGS. 3A and B). CDK1/Cyclin B indeed phosphorylated CDKACTH1, however in the same conditions as CDK2/Cyclin A it did not induce the same response. However, characterization of sensor and of kinase dose-dependency, respectively, revealed that the optimal response was achieved at 10 nM sensor and 50-100 nM CDK1/Cyclin B (FIGS. 9A-D). Dose-dependency of CDK1/Cyclin B showed that optimal response for this kinase was obtained at different concentrations from those determined for CDK2/Cyclin A, with 10 nM CDKACTH1 sensor and 100 nM kinase (FIGS. 9A-D). Likewise, CDK4/Cyclin D kinase induced changes in CDKACTH1 fluorescence reminiscent of CDK2/Cyclin A. The signal was dependent on MgCl2/ATP, and was further affected by addition of the PD-0332991 inhibitor, whereas addition of roscovitine had no effect. Plk1 or Plk3 did not induce any significant changes in the fluorescence of CDKACTH1, in the same range of concentrations (FIG. 3C-E).

These results show that a biosensor according to the invention allows to determine enzymatic activity profiles of CDK/cyclin and to compare the efficiency of inhibitors that interfere with kinase activity.

### Example 3

### Application of CDKACT-H1 to Monitor CDK/Cyclin Activity in Cell Extracts

We next asked whether CDKACTH1 could be applied to probe CDK/Cyclin kinase activity in cell extracts.

### Cell Culture and Extract Preparation

Cell culture media, serum and antibiotics were purchased from Invitrogen. HeLa, HS68, A549, MCF-7 and U2OS cells were cultured in DMEM+Glutamax supplemented with 10% FCS, 1 mM penicillin and 1 mM streptomycin at 37° C. in an atmosphere containing 5% CO2. HT2-19 cells, provided by Dr. A. Porter, were cultured in DMEM+NEAA, antibiotics, sodium pyruvate and glutamine, and supplemented with 10% FCS29. Cell extracts were prepared in TBS lysis buffer containing 50 mM TrisHCl, pH 7.4, 150 mM NaCl, 0.1% NP40, 0.1% Deoxycholate, 2 mM EDTA, 1 mM PMSF, Complete™ protease inhibitors (Roche), and normalized following spectrophometric dosage at 280 nm. Phosphatase inhibitors: 50 mM NaF, 40 mM β-Glycero-phosphate, 1 mM Na3VO4.

For enrichment of cellular CDK/Cyclin complexes, HeLa cell extracts prepared in TBS lysis buffer were separated on a Superose 12 FPLC column in TBS buffer and immediately used for fluorescence-bad kinase assays or for Western blotting.

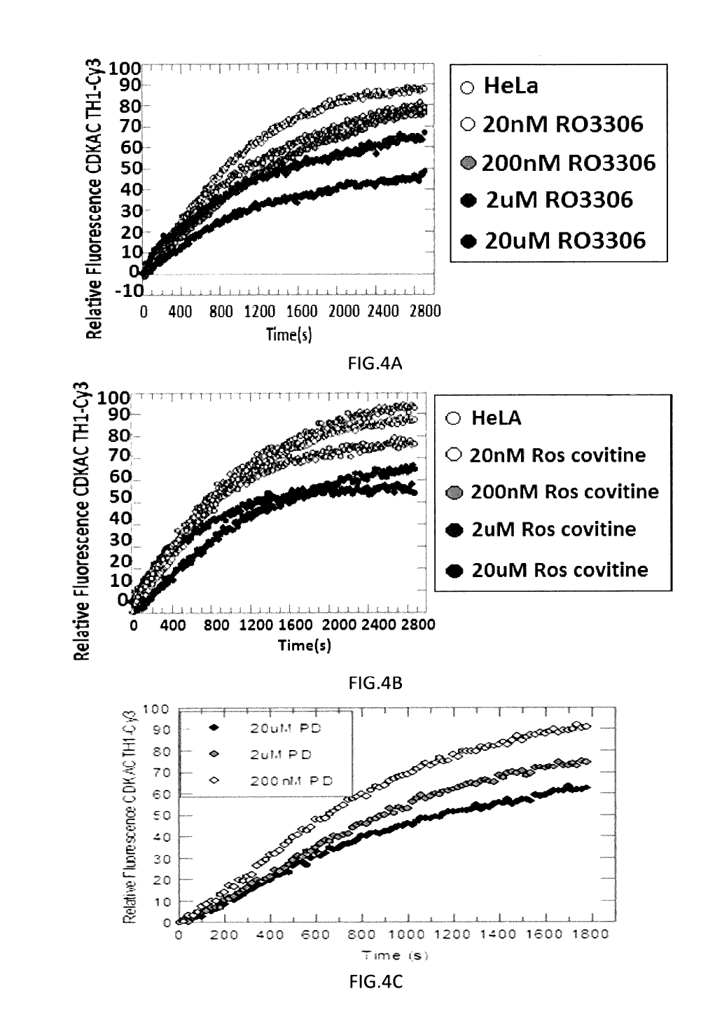

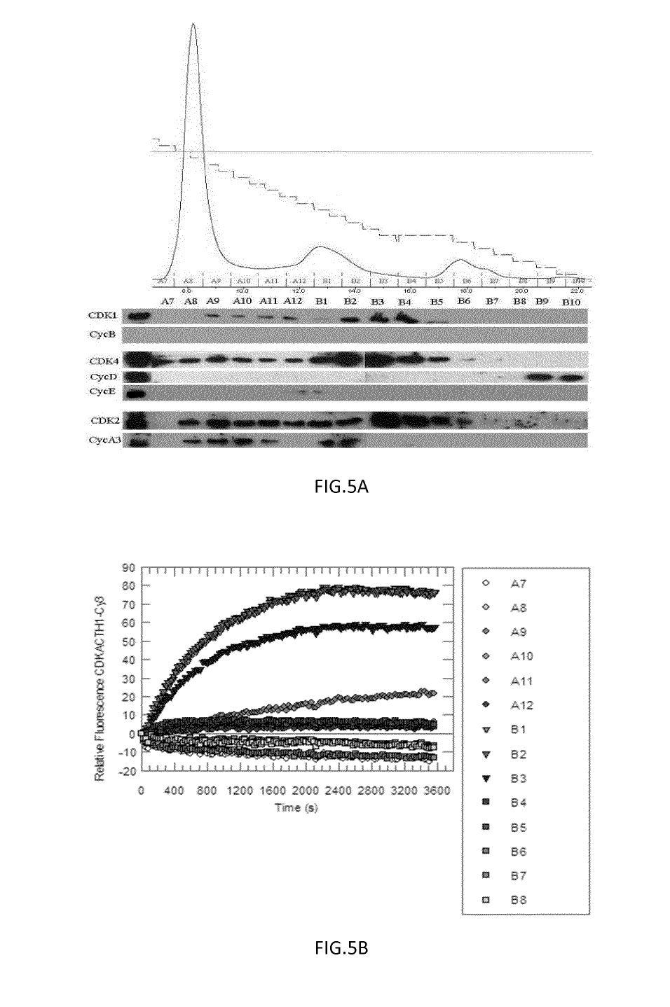

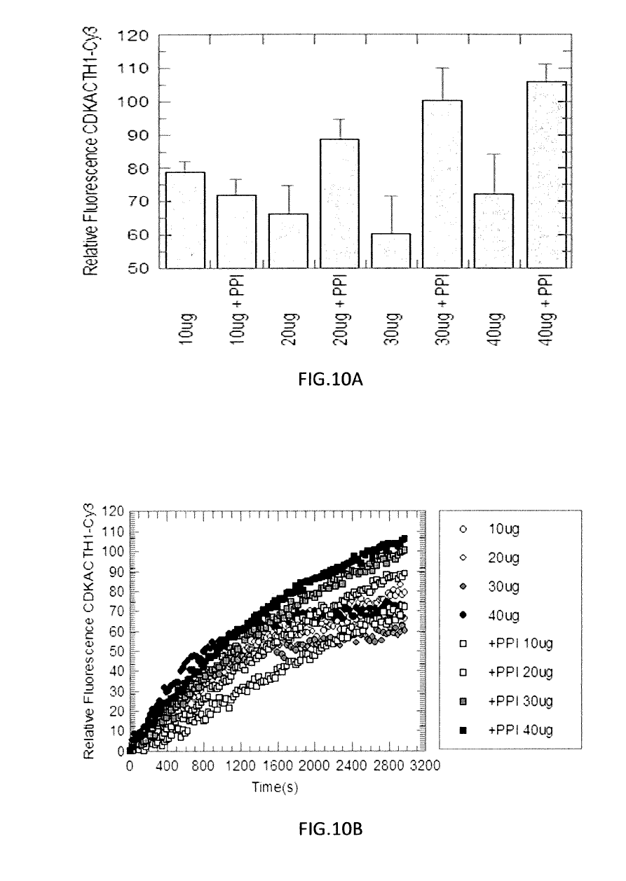

Kinase assays were performed in the same conditions as above. These experiments revealed that 20 ug lysates were ideal to probe CDKACTH1 activity, the signal reaching saturation within a couple of minutes. As shown in FIG. 4A-C, incubation of 20 ug HeLa cell extracts with CDKACTH1 yielded a very similar profile to that obtained with recombinant CDK/Cyclins, implying that these kinases were indeed phosphorylating the substrate sequence of the biosensor. In order to verify the specificity of this reaction, the assay was performed in the presence of several CDK/Cyclin inhibitors: roscovitine, R03306 and PD-0332991, as well as with DMSO as a negative control (FIG. 4A-C). These experiments clearly revealed that the fluorescence enhancement of CDKACTH1 was associated with CDK/Cyclin activity. The experiment with the PD inhibitor showed that CDK4/CyclinD was responsible for partial activity, the experiment with RO3306 inhibitor and roscovitine showed that CDK1 and CDK2 activities were responsible for the larger part of activities. Since phosphorylation events are counterbalanced by phosphatases in mammalian cell extracts, we asked whether the response measured would be further improved if phosphatase activity was blocked. Indeed, addition of phosphatase inhibitors increased the fluorescence signal by 10% (FIGS. 10A-10B).

To further validate the fact that the response measured was due to CDK/Cyclin kinase activity, we enriched the CDK/Cyclins by size exclusion chromatography of the cell lysates and performed both a kinase assay with CDKACTH1 sensor and a Western blot of the different fractions (FIGS. 5A-5B). We found that fractions B1-B3 contained very high kinase activity, whereas all fractions before or after these did not show any significant activity. In line with these results, Western blot analysis revealed that fractions B1-B3 contained high levels of both CDKs and Cyclins.

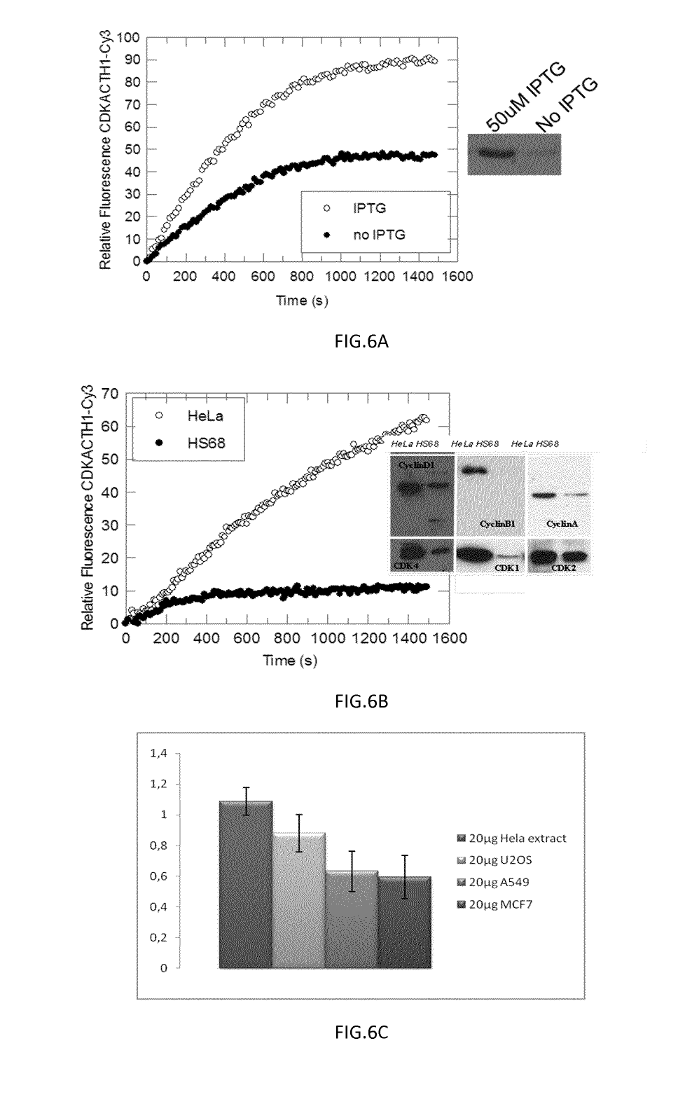

We finally applied CDKACTH1 to compare the activity of CDK/Cyclins in cell extracts expressing different levels of CDKs and Cyclins (FIG. 6A-C). We first applied CDKACTH1 to monitor kinase activity of the HT2-19 cell line, in which the levels of CDK1 can be modulated by addition of IPTG to induce ectopical expression of a single CDK1 allele (Itzhaki el al. Nat. Genetics (1997) 15: 258-265). We have previously shown that when these cells are grown in the absence of IPTG for 4 days, CDK1 levels are reduced by 90% compared to cells grown with IPTG (Kurzawa et al. PloS One (2011) 6(10):e26555). 20 ug total extracts prepared from HT2-19 cells grown in these two conditions were assayed in a fluorescence-based kinase assay with CDKACTH1, revealing reduction of kinase activity by 50%, inferring that in this cell line, CK1 activity is responsible for half of all CDK/Cyclin kinase activity (FIG. 6A). We next compared overall CDK/Cyclin kinase activity between the fibroblastic cell line HS68 and the cervical cancer cell line HeLa. When using identical concentrations of total cell extracts, we observed a very weak kinase activity in HS68 cells compared to HeLa, in agreement with Western blot analysis of total CDK and Cyclin levels (FIG. 6B). Finally we compared the average CDK/Cyclin kinase activity in different cancer cell lines following normalization of their total protein levels. In this assay we compared HeLa cell extracts which we used as a standard with U20S, A549 and MCF-7 cells. These data provide a readout of total CDK/Cyclin kinase activity between these cell lines (FIG. 6C).

Therefore, a biosensor according to the invention allows to report differences associated with expression levels of CDKs and cyclins, and to compare CDK/cyclin activities in extracts from different healthy and cancer cell lines.

### Example 4

### Application of CDKACT-H1 to Monitor CDK/Cyclin Activity in Living Cells

Finally we applied CDKACTH1 to monitor CDK/Cyclin kinase activity in living cells. To this aim, we engineered an RFP-CDKACTH1 construct, which was expressed in E. coli and purified by FPLC, then labeled with a Cy5 probe and further purified from the free label. This fluorescent protein biosensor was then complexed to the cell-penetrating peptide Pep1 at a 20:1 molar ratio, and then incubated with HeLa cells to allow for efficient cellular uptake (Morris et al. Nat. Biotechnol. (2001) 19, 1173-1176).

### Time-Lapse Imaging and Fluorescence Quantification

Live-cell imaging acquisitions of RFP and Cy5 fluorescence associated with RFP-CDKACTH1-Cy5 in living cells were substracted for background signal corresponding to minimal fluorescence levels using Metamorph. Image J was then used for image analysis. Regions of interest (ROI) corresponding to 10-15 cells in which fluorescence appeared homogeneous were designed and the mean grey levels of fluorescence for each channel (RFP and Cy5) were quantified within these ROIs for each experiment, and the mean ratiometric value of Cy5/RFP was calculated from 3-4 different ROIs.

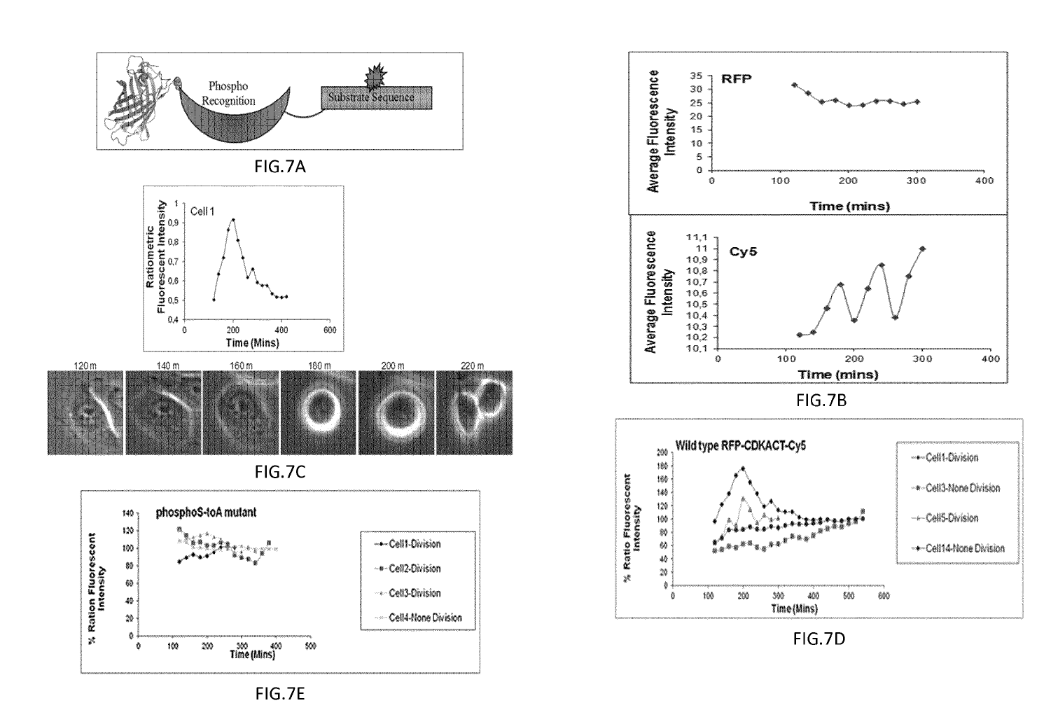

Following internalization, cells were imaged by time-lapse microscopy and the fluorescence signal corresponding to RFP and to Cy5, were acquired separately, then normalized and used for ratiometric quantification of Cy5/RFP. As shown in FIG. 7B, in a field of asynchronous, cycling HeLa cells, several peaks of Cy5 fluorescence, corresponding to kinase activity detected by CDKACTH1 were observed, whilst RFP signal was essentially flat, following an initial period (120 min) of quenching, attributable to the release of the RFP-biosensor from the cell-penetrating peptide complex particle. To further address whether this signal was attributable to kinase activity associated with cell division, we chose individual cells that were undergoing mitosis, and compared the Cy5/RFP signal to that of cells that were not dividing over the duration of the time-lapse (FIG. 7D). In these experiments we found that a fluorescent peak could only be observed in dividing cells. Moreover, when we used a non-phosphorylatable mutant of CDKACTH1 in which the phosphorylatable threonine within the substrate domain was mutagenized to an alanine, we no longer observed peaks of Cy5/RFP, even in cells that were undergoing mitosis (FIG. 7E).

These results indicate that RFP-CDKACT-Cy5 biosensor allows to monitor periodic oscillations associated with CDK/Cyclin activity at the onset of mitosis.

### Example 5

### In Vitro Characterization of CDKACT-WWH1 Peptide Biosensor and Comparison with CDKACTH1 Biosensor

The CDKACT-WWH1 biosensor (SEQ ID N^{o} 20) comprises, from its Nt to its Ct extremity, a phosphobinding domain derived from Pin1 WW (SEQ ID N^{o} 10), a linker (SEQ ID N^{o} 14) and a substrate domain derived from Histone H1 (SEQ ID N^{o} 12). The CDKACT-WWH1 biosensor was synthesized, then labeled with Cy5 as described in Example 1.

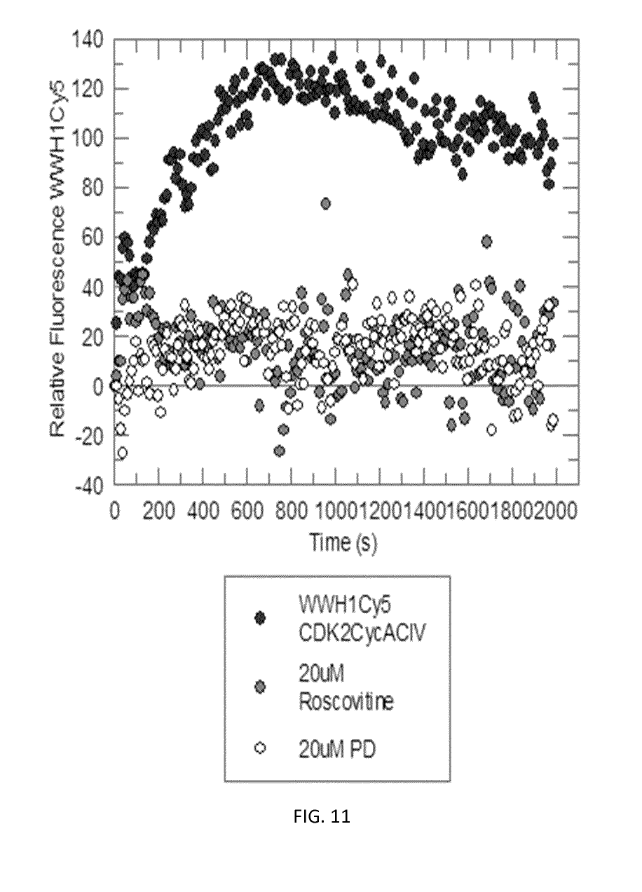

In vitro characterization was performed as described in Example 2. Fluorescence intensity assays were performed in 96-well plates, in 200 ul with 25 nM Cy5-CDKACT-WWH1+/−25 nM active CDK2/Cyclin A3 in the presence of 5 mM ATP& 20 mM MgCl2+/−20 uM Roscovitine or PD0332991 CDK/Cyclin Inhibitors (FIG. 11). Assays were performed at 30° C. for 1600s. Fluorescence emission of Cy5 was measured at 675 nm following excitation at 645 nm on Polarstar™ fluorimeter.

### Conclusion:

The peptide version of CDKACTH1, CDKACTWWH1, reports on CDK2/CyclinA activity, through significant changes in fluorescence enhancement.

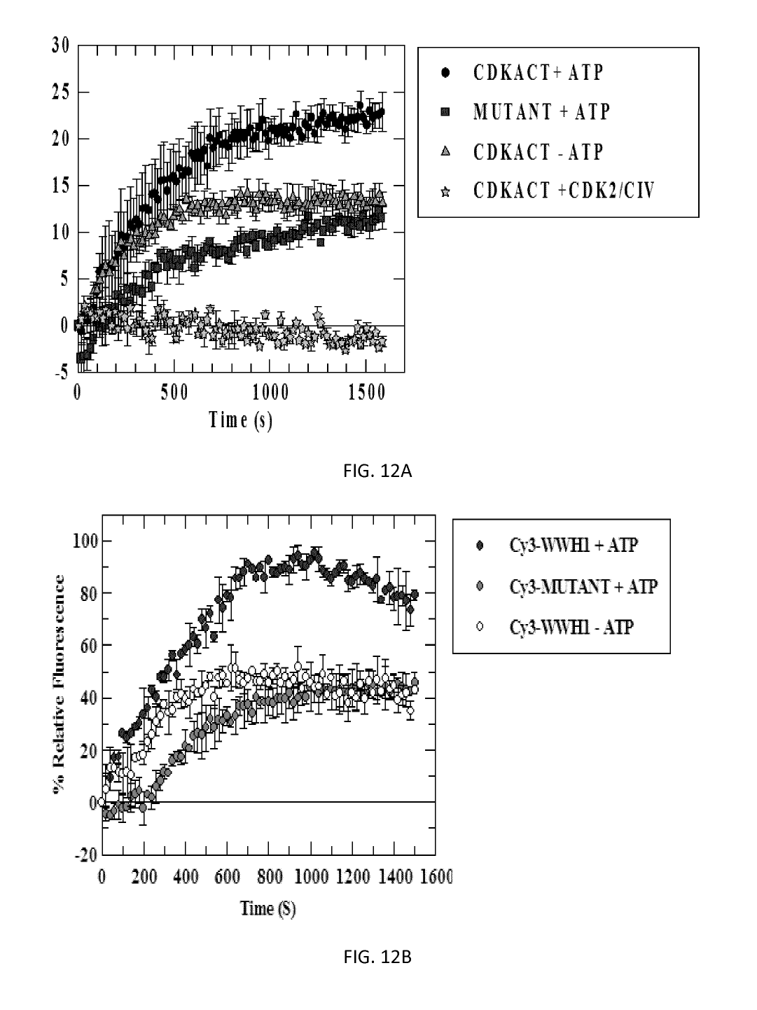

In order to compare the binding of CDKACTH1 and of CDKACTWWH1, fluorescence intensity assays were performed in 96-well plates, in 200 ul with 25 nM Cy3-CDKACTH1 or Cy3-CDKACT-WWH1 or their non phosphorylatable mutants, in which the threonine phosphorylation site was replaced by an alanine, in the presence or not of 25 nM active CDK2/Cyclin A3 in the presence of 5 mM ATP and 20 mM MgCl2 (FIGS. 12A and 12B). Assays were performed at 30° C. for 1600s. Fluorescence emission of Cy3 was measured at 590 nm following excitation at 544 nm on Polarstar™ fluorimeter.

### Conclusion:

This assay allows to distinguish the contribution due to kinase binding and to the phosphotransfer reaction itself. Both CDKACTH1 and CDKACTWWH1 report on CDK2/CyclinA activity, however CDKACTWWH1 yields greater changes in fluorescence enhancement. Moreover, whereas CDKACTH1 is easy to produce as a recombinant protein, and useful for a daily laboratory use, CDKACTWWH1 may be more suitable for in vivo experiments.

### Example 6

### In Vivo Use of CDKACT Biosensor for Detection of CDK/Cyclins

3 million A375 rvluc2 cells were implanted subcutaneously into NMRI nude mice and grown for 15 days then not treated (control) or treated with DMSO (vehicle) or Roscovitine (400 μL IP, 50 mg/kg/day for 7 days). After 20 days, tumours were injected intratumorally with 100 ul 50/50 6 uM PEP-CDKACT-Cy5/6 uM PEP-Ctrl-Alexa 750.

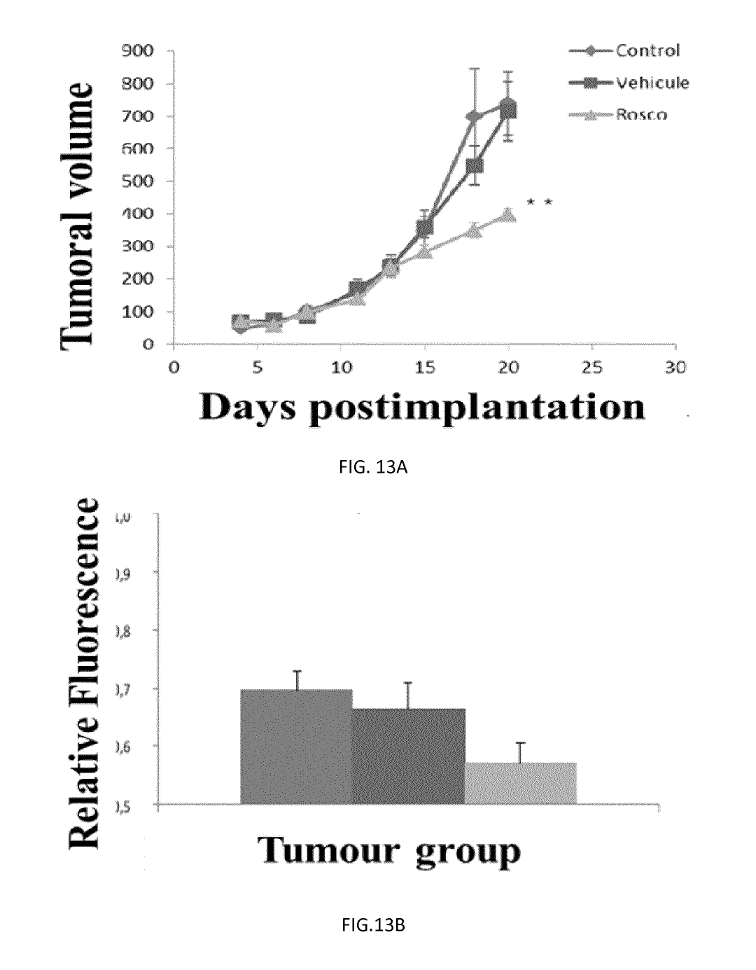

The size of tumours treated with Roscovitine is statistically smaller (53.8% compared to control; 55.6% compared to vehicle (FIG. 13A). The CDKACT-Cy5/Ctrl-Alexa750 fluorescence in the three tumour groups in shown in FIG. 13B.

Conclusion: The autopenetrating PEP-CDKACT-Cy5 biosensor allows to image differences in CDK/Cyclin activity in tumour xenografts that correlate with the difference in tumour growth upon administration of a CDK/Cyclin inhibitor (Roscovitine)

### BIBLIOGRAPHIC REFERENCES

1. Malumbres et al., Trends Biochem Sci., 30:630-641, 2005
2. Obaya et al., Cell Mol Life Sci., 59:126-142, 2002
3. Satyanarayana et al. Oncogene. 28:2925-2939, 2009
4. Merrick et al., Cell Cycle. 9:706-714, 2010
5. Jeffrey et al., Nature, 376:313-320, 1995
6. Morgan et al., Annu Rev Cell Dev Biol., 13:261-291, 1997
7. Morris et al. J Biol Chem., 277:23847-23853, 2002
8. Lolli et al., Cell Cycle; 9:1551-1561, 2010
9. Morgan et al., Annu Rev et al., Annu Rev Cell Dev Biol., 13:261-291, 1997
10. Lapenna et al., Nat Rev Drug Discovery, 8:547-566, 2009
11. Malumbres et al., Nat Rev Cancer., 9:153-166, 2009
12. Harwell et al., J Biol Chem., 279:12695-12705, 2004
13. Ekberg et al., Eur J Haematol., 75:106-115, 2005
14. Husdal et al., Cell Oncol., 18:107-116, 2006
15. Suzuki et al., Cancer Sci., 98:644-651, 2007
16. Kim et al., J Cell Physiol. 220:292-296, 2009
17. Stivala et al., Curr Cancer Drug Targets, 12(2):85-96, 2012
18. Nozoe et al., Oncology, 71(5-6):402-10, 2006
19. Malumbres et al., Rev Cancer., 1:222-231, 2001
20. Malumbres at al., Curr Opin Genetics and Development, 17:60-65, 2007
21. Versteeg et al., Biochem. J., 350: 717-720, 2000)
22. Kupcho et al., Anal Biochem., 317(2):210-7, 2003
23. Loving et al., Trends Biotechnol., 28(2):73-83, 2010
24. Lowder et al., Curr. Opin. Chem. Biol., 15(6):781-8, 2011
25. Kurzawa et al. Biochim. Biophys. Acta 1798:2274-2285, 2010
26. Morris et al. Nat. Biotechnol. 19: 1173-1176, 2001
27. Grdisa et al., Curr. Med. Chem., 18(9): 1373-1379, 2011
28. Matjaz et al., Adv. Drug Deliv. Rev., 57(4): 529-545, 2005
29. Morris et al. Biol. Cell, 100: 201-217, 2008
30. Fonseca et al. Advanced Drug Delivery Reviews, 61: 953-964, 2009
31. Heitz et al. British J. Pharmacol. 157:195-206, 2009
32. Heitz et al., Biochemistry. 36, 4995-5003, 1997
33. Itzhaki el al. Nat. Genetics (1997) 15: 258-265
34. U.S. Pat. No. 5,730,969 “Method and compositions for solubilization and stabilization of polypeptides,” Hora et al., 1998
35. U.S. Pat. No. 5,190,762 “Method of administering proteins to living skin cells”, Yarosh et al., 1993

1. A compound comprising a polypeptide and at least one fluorophore, characterized in that:
said polypeptide comprises:
a substrate domain comprising the sequence X₁-S₁-S₂/T-P-X₂ SEQ ID NO:30 and capable of being phosphorylated by a CDK and/or a cyclin-CDK complex on the amino acid residue at position 3 of said sequence SEQ ID NO:30,
a phosphobinding domain,
wherein said phosphobinding domain and said substrate domain are capable of interacting upon the phosphorylation of said ST amino-acid residue and/or of said T amino-acid residue of said sequence X₁-S₁-S₂/T-P-X₂ in the substrate domain,
wherein the substrate domain and the phosphobinding domain are linked by a linker domain, and
said at least one fluorophore is coupled to an amino-acid residue of said substrate domain.

2. A compound according to claim 1 characterized in that X₁ and/or X₂ is a cysteine.

3. A compound according to claim 1, characterized in that the amino acid sequence of the substrate domain comprises at least one sequence chosen from the list consisting of:
SEQ ID NO: 1
a. GGCSTPKKAKKL [[(SEQ ID No 1)]]
SEQ ID NO: 2
b. PEPILVDCSSPSPMET [[(SEQ ID No 2)]]
SEQ ID NO: 3
c. RAGGPATCSSPTRL [[(SEQ ID No 3)]]
SEQ ID NO: 4
d. YKFCSSPLRIPG [[(SEQ ID No 4)]]
SEQ ID NO: 5
e. SGYSSPGSCSTPGSR [[(SEQ ID No 5).

4. A compound according to claim 1, characterized in that the amino acid sequence of the phosphobinding domain comprises a sequence derived from a sequence chosen in the group consisting of: the sequence of Plk1 SEQ ID NO:6, the sequence of Pin1 SEQ ID NO:7, the sequence of Chk2 SEQ ID NO:8.

5. A compound according to claim 1, characterized in that the amino-acid sequence of said phosphobinding domain comprises a sequence chosen from the list consisting of: the sequence SEQ ID NO:10, the sequence SEQ ID NO:11, the sequence SEQ ID NO:12.

6. A compound according to claim 1, characterized in that said fluorophore is coupled to a cysteine.

7. A compound according to claim 1, characterized in that said fluorophore is an environmentally-sensitive dye.

8. A compound according to claim 1, characterized in that it comprises at least a couple of fluorophores capable of FRET.

9. A compound according to claim 1, wherein the amino acid sequence of said polypeptide is chosen from the list consisting of: the sequence SEQ ID NO:20, the sequence SEQ ID NO:21, the sequence SEQ ID NO:22, the sequence SEQ ID NO:23, the sequence SEQ ID NO:24, the sequence SEQ ID NO:25, the sequence SEQ ID NO:26, the sequence SEQ ID NO:27, the sequence SEQ ID NO:28, the sequence SEQ ID NO:29, the sequence SEQ ID NO:31, the sequence SEQ ID NO:32, the sequence SEQ ID NO:33, the sequence SEQ ID NO:34, the sequence SEQ ID NO:35, and wherein the fluorophore is Cy3 and is coupled to a cysteine residue of said polypeptide.

10. A compound according to claim 1, characterized in that it further comprises a cell-penetrating peptide sequence and/or a protein tag.

11. A composition comprising at least one compound according to claim 1 and a pharmaceutically acceptable carrier.

12. Use of at least one compound according to claim 1 or a composition according to claim 11 for fluorescence imaging.

13. A method for determining if at least one CDK and/or one Cyclin-CDK complex is active, comprising the steps of:
a) providing at least one compound according to claim 1
b) contacting said compound with said CDK and/or Cyclin-CDK complex,
c) illuminating said compound and said CDK and/or Cyclin-CDK complex with an excitation light,
determining the fluorescence signal emitted by said compound,
e) comparing said fluorescence signal with a reference fluorescence signal, and
f) determining from the comparison of step e) if at least one CDK and/or Cyclin-CDK complex is active.

14. A method for determining the kinetics of at least one CDK and/or one Cyclin-CDK complex activity, comprising the steps of:
a) providing at least one compound according to claim 1,
b) contacting said compound with said CDK and/or Cyclin-CDK complex,
c) illuminating said compound and said CDK and/or Cyclin-CDK complex with an excitation light,
d) determining the fluorescence signal emitted by said compound,
e) repeating steps c) and d) at a different times, at least once,
f) comparing said fluorescence signals of step d) and step e), and
g) determining, from the comparison of step f), the kinetics of at least one CDK and/or Cyclin-CDK complex activity.

15. A method for the in vitro diagnosis of CDK and/or Cyclin-CDK complex hyperactivation in a subject, comprising the steps of:
h) providing at least one compound according to claim 1,
i) contacting said compound with a biological sample from said subject,
j) illuminating said biological sample and compound with an excitation light,
k) determining the fluorescence signal emitted by said compound,
l) comparing said fluorescence signal with a reference fluorescence signal, and
m) determining from the comparison of step e) if at least one CDK and/or Cyclin-CDK complex is hyperactive.


SEQUENCE LISTING (35 sequences)

SEQ ID NO: 1 - protein, 12 aa, artificial
  Substrate domaine Histone H1
GGCSTPKKAKKL

SEQ ID NO: 2 - protein, 16 aa, artificial
  Substrate domaine Cyclin B1
PEPILVDCSSPSPMET

SEQ ID NO: 3 - protein, 14 aa, artificial
  Substrate domain Lamin B2
RAGGPATCSSPTRL

SEQ ID NO: 4 - protein, 12 aa, artificial
  Substrate domain Retinoblastoma protein
YKFCSSPLRIPG

SEQ ID NO: 5 - protein, 15 aa, artificial
  Substrate domain Tau
SGYSSPGSCSTPGSR

SEQ ID NO: 6 - protein, 603 aa, homo sapiens
MSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 7 - protein, 162 aa, homo sapiens
MADEEKLPPGWEKRMSRSSGRVYYFNHITNASQWERPSGNSSSGGKNGQGEPARVRCSHLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

SEQ ID NO: 8 - protein, 543 aa, homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 9 - protein, 237 aa, artificial
  PhosphoBindingDomain of PLK1
GEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 10 - protein, 34 aa, artificial
  WW domain of PIN1
KLPPGWEKRMSRSSGRVYYFNHITNASQWERPSG

SEQ ID NO: 11 - protein, 63 aa, artificial
  FHA domain of Chk2
YWFGRDKSCEYCFDEPLLKRTDKYRTYSKKHFRIFREVGPKNSYIAYIEDHSGNGTFVNTELV

SEQ ID NO: 12 - protein, 237 aa, artificial
  Phosphobinding domain of Plk1 protein wherein all Cystein residues are replaced by a Serine residue
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKAS

SEQ ID NO: 13 - protein, 63 aa, artificial
  FHA domain of Chk2 protein wherein all Cystein residues are replaced by a Serine residue
YWFGRDKSSEYSFDEPLLKRTDKYRTYSKKHFRIFREVGPKNSYIAYIEDHSGNGTFVNTELV

SEQ ID NO: 14 - protein, 12 aa, artificial
  Linker Sequence
PGAGGTGGLPGG

SEQ ID NO: 15 - protein, 258 aa, artificial
  Polypeptide comprising: the phosphobinding domain of Plk1 wherein all Cystein residues are replaced by a Serine residue, a linker and the CDK substrate domain from histone H1
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSELLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGCSTPKKAKKL

SEQ ID NO: 16 - protein, 264 aa, artificial
  Polypeptide comprising: the phosphobinding domain of Plk1 wherein all Cystein residues are replaced by a Serine residue, a linker and the CDK substrate domain from cyclin B1
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSELLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGPEPILVDCSSPSPMET

SEQ ID NO: 17 - protein, 262 aa, artificial
  Polypeptide comprising: the phosphobinding domain of Plk1 wherein all Cystein residues are replaced by a Serine residue, a linker and the CDK substrate domain from Lamin B2
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSELLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGRAGGPATCSSPTRL

SEQ ID NO: 18 - protein, 260 aa, artificial
  Polypeptide comprising: the phosphobinding domain of Plk1 wherein all Cystein residues are replaced by a Serine residue, a linker and the CDK substrate domain from Retinoblastoma
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSELLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGYKFCSSPLRIPG

SEQ ID NO: 19 - protein, 263 aa, artificial
  Polypeptide comprising: the phosphobinding domain of Plk1 wherein all Cystein residues are replaced by a Serine residue, a linker and the CDK substrate domain from Tau
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSELLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGSGYSSPGSCSTPGSR

SEQ ID NO: 20 - protein, 58 aa, artificial
  Polypeptide comprising: the WW domain of PIN1 wherein all Cystein residues are replaced by a Serine residue, a linker and the CDK substrate domain from histone H1
KLPPGWEKRMSRSSGRVYYFNHITNASQWERPSGPGAGGTGGLPGGGGCSTPKKAKKL

SEQ ID NO: 21 - protein, 62 aa, artificial
  Polypeptide comprising: the WW domain of PIN1 wherein all Cystein residues are replaced by a Serine residue, a linker and the CDK substrate domain from cyclin B1
KLPPGWEKRMSRSSGRVYYFNHITNASQWERPSGPGAGGTGGLPGGPEPILVDCSSPSPMET

SEQ ID NO: 22 - protein, 60 aa, artificial
  Polypeptide comprising: the WW domain of PIN1 wherein all Cystein residues are replaced by a Serine residue, a linker and the CDK substrate domain from Lamin B2
KLPPGWEKRMSRSSGRVYYFNHITNASQWERPSGPGAGGTGGLPGGRAGGPATCSSPTRL

SEQ ID NO: 23 - protein, 58 aa, artificial
  Polypeptide comprising: the WW domain of PIN1 wherein all Cystein residues are replaced by a Serine residue, a linker and the CDK substrate domain from Retinoblastoma
KLPPGWEKRMSRSSGRVYYFNHITNASQWERPSGPGAGGTGGLPGGYKFCSSPLRIPG

SEQ ID NO: 24 - protein, 61 aa, artificial
  Polypeptide comprising: the WW domain of PIN1 wherein all Cystein residues are replaced by a Serine residue, a linker and the CDK substrate domain from Tau
KLPPGWEKRMSRSSGRVYYFNHITNASQWERPSGPGAGGTGGLPGGSGYSSPGSCSTPGSR

SEQ ID NO: 25 - protein, 87 aa, artificial
  Polypeptide comprising: the FHA domain of Chk2 wherein all Cystein residues are replaced by a Serine residue, a linker and the CDK substrate domain from histone H1
YWFGRDKSSEYSFDEPLLKRTDKYRTYSKKHFRIFREVGPKNSYIAYIEDHSGNGTFVNTELVPGAGGTGGLPGGGGCSTPKKAKKL

SEQ ID NO: 26 - protein, 91 aa, artificial
  Polypeptide comprising: the FHA domain of Chk2 wherein all Cystein residues are replaced by a Serine residue, a linker and the CDK substrate domain from cyclin B1
YWFGRDKSSEYSFDEPLLKRTDKYRTYSKKHFRIFREVGPKNSYIAYIEDHSGNGTFVNTELVPGAGGTGGLPGGPEPILVDCSSPSPMET

SEQ ID NO: 27 - protein, 89 aa, artificial
  Polypeptide comprising: the FHA domain of Chk2 wherein all Cystein residues are replaced by a Serine residue, a linker and the CDK substrate domain from Lamin B2
YWFGRDKSSEYSFDEPLLKRTDKYRTYSKKHFRIFREVGPKNSYIAYIEDHSGNGTFVNTELVPGAGGTGGLPGGRAGGPATCSSPTRL

SEQ ID NO: 28 - protein, 87 aa, artificial
  Polypeptide comprising: the FHA domain of Chk2 wherein all Cystein residues are replaced by a Serine residue, a linker and the CDK substrate domain from Retinoblastoma
YWFGRDKSSEYSFDEPLLKRTDKYRTYSKKHFRIFREVGPKNSYIAYIEDHSGNGTFVNTELVPGAGGTGGLPGGYKFCSSPLRIPG

SEQ ID NO: 29 - protein, 90 aa, artificial
  Polypeptide comprising: the FHA domain of Chk2 wherein all Cystein residues are replaced by a Serine residue, a linker and the CDK substrate domain from Tau
YWFGRDKSSEYSFDEPLLKRTDKYRTYSKKHFRIFREVGPKNSYIAYIEDHSGNGTFVNTELVPGAGGTGGLPGGSGYSSPGSCSTPGSR

SEQ ID NO: 30 - protein, 5 aa, artificial
  Consensus phosphorylated sequence from the CDK substrate domain
  misc_feature (1)..(1): Xaa can be any naturally occurring amino acid
  VARIANT (3)..(3): Ser may be replaces by Thr
  misc_feature (5)..(5): Xaa can be any naturally occurring amino acid
XSSPX

SEQ ID NO: 31 - protein, 261 aa, artificial
  Polypeptide comprising: the phosphobinding domain of Plk1 wherein all Cystein residues are replaced by a Serine residue, a linker and the CDK substrate domain from histone H1
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGGGCSTPKKAKKL

SEQ ID NO: 32 - protein, 265 aa, artificial
  Polypeptide comprising: the phosphobinding domain of Plk1 wherein all Cystein residues are replaced by a Serine residue, a linker and the CDK substrate domain from cyclin B1
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSHELLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGPEPILVDCSSPSPMET

SEQ ID NO: 33 - protein, 263 aa, artificial
  Polypeptide comprising: the phosphobinding domain of Plk1 wherein all Cystein residues are replaced by a Serine residue, a linker and the CDK substrate domain from Lamin B2
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGRAGGPATCSSPTRL

SEQ ID NO: 34 - protein, 261 aa, artificial
  Polypeptide comprising: the phosphobinding domain of Plk1 wherein all Cystein residues are replaced by a Serine residue, a linker and the CDK substrate domain from retinoblastoma
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGYKFCSSPLRIPG

SEQ ID NO: 35 - protein, 264 aa, artificial
  Polypeptide comprising: the phosphobinding domain of Plk1 wherein all Cystein residues are replaced by a Serine residue, a linker and the CDK substrate domain from Tau
GEVVDSHLSDMLQQLHSVNASKPSERGLVRQEEAEDPASIPIFWVSKWVDYSDKYGLGYQLSDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILSPLMAAVTYIDEKRDFRTYRLSLLEEYGSSKELASRLRYARTMVDKLLSSRSASNRLKASPGAGGTGGLPGGSGYSSPGSCSTPGSR